SEQUENCE LISTING (80 sequences)

SEQ ID NO: 1 - DNA, 509 bp, Drosophila melanogaster
gatagattcgtggccatctgtaacccattgttatactcagttgctatgtcccagaggctctgcatccagctagtggtgggtccctatgtcattggactcatgaataccatgactcacacaacaaatgcattttgtctccctttttgtggccctaatgtcatcaatcctttcttctgtgatatgtcccccttcctttcccttgtatgtgctgataccaggctcaataagttggcagttttcatcgtggctggagctgtgggagtcttcagtggcccgactatcctgatttcctacatttacatcctcatggccatcctgaggatgtccgctgatgggaggtgcagaaccttttctacttgctcttctcacccgacagctgctttcatctcgtatggtaccctcttctttatttatgtacatcccagtgcaaccttctccctggatctcaataaagtagtgtctgtgttttacacagcagtgattcccatgctcaaccccttcatctgcag

SEQ ID NO: 2 - DNA, 264 bp, Drosophila melanogaster
gcaataaatacagacaattatatatatttttctataatttgtttcattttatcattttattgattgtgaacgaaaaaggaaaaaaataaacttagaaaaagataacaaataaattgctaaatttaagcgcaaaagttcaattaataaaaattagaattttaatactaacataatttggactatttatatatacatacgcatatatatacaactctatatatgaataattagtaacaaatcaaatcaatttccataacaaccgct

SEQ ID NO: 3 - DNA, 367 bp, Drosophila melanogaster
tcttttttgaattttattcaattttaaagtacaattgcacgagtgatttttgttgcactcgtcactaagaatatatatatatttttttgtttttttttctggcggttgttatggaaattgatttgatttgttactaattattcatatatagagttgtatatatatgcgtatgtatatataaatagtccaaattatgttaatattaaaattctaatttttattaattgaacttttgcgcttaaatttagcaatttatttgttatctttttctaagtttatttttttcctttttcgttcacaatcaataaaatgataaaatgaaacaaattatagaaaaatatatataattgtctgtatttattgcgga

SEQ ID NO: 4 - DNA, 483 bp, Drosophila melanogaster
  misc_feature (1)...(483): n = A,T,C or G
ttggtttgataatgntcntactccaaaaacaaaacaataatttatactggtcntgnttgnttgtcataacntcacagtagtatactcgntcnttttggtaataatcccgaaaaaactgnaccgaatccagnaanatatccccgtcaanaaaaaaaacatataaaatatgaaatggtacataanaaatatgtccantccaaccaaccaaccaaacaaccaaccaacaaacaacaannaccaaccaacccaaaatcccaaataaccgccaacaatccaaaatggtaactaaaaccattggtgaaaacaggatacaagccacttatcctaacaaacgccaggctacactgagaaaataagcatcgngagttggtatggatagcagaaattacccatattcgtggactaaaggtggtgtactgattgactgattgattgacgtgttgggtggtgaatacatatatttcgactgcatgccaaggaata

SEQ ID NO: 5 - DNA, 395 bp, Drosophila melanogaster
tgtttgtatgtctactccaaaaacaaaacaataatttatactgtctgttgttgtcataactcacatatatactcgtcttttgtatatccgaaaaactgaccgatccaaaaatatccccgcaaaaaaaaacatataaaatatgaatgtacataaaaatatgtccatccaaccaaccaaccaaacaaccaaccaacaaacaacaaaaccaaccaacccaaaatcccaaaaaccgccaacaatccaaaatgtaactaaaaccattgtgaaaacagatacaagccacttatcctaacaaacgccaggctacactgagaaaataagcatcggagttggtatggatagcagaaattacccatattcgtggactaaaggtggtgtactgattgactgattga

SEQ ID NO: 6 - DNA, 188 bp, Drosophila melanogaster
agtattttctttagtttctttgaggtgtgttagcacactcattgttgctttagcctagcgctggtttattaaatgttagctaagtttaaattatgtatttacagatgctgtgtgctagctcgaaagtgataatttsgtgttattttttgtgtatgggattttgataaatgccttatgagtttagaacg

SEQ ID NO: 7 - DNA, 186 bp, Drosophila melanogaster
agtattttctttagtttctttgaggtgtgttagcacactcattgttgctttagcctagcgctggtttattaaatgttagctaagtttaaattatgtatttacagatgctgtgtgctagctcgaaagtgataatttgtgttattttttgtgtatgggattttgataaatgccttatgagtttagaac

SEQ ID NO: 8 - DNA, 297 bp, Drosophila melanogaster
acatttccatggtttattttaatgtgaagttaaactgcaaatttctagtctaagcgtagtagttaagattagccttcttcttcgcctgcacttccatgatggcgtccatgaagtcttcgtgtgtaaccgagttggcggagcgacgcagtgcgatcataccagcttccacacagacggctttgcactgggcgccgttgaagtcatccgtggatcgggacaattcctcgaaattcacatcattgctaacgttcattttacgcgagtgaatctgcataatacgggcacgggcttcctcgt

SEQ ID NO: 9 - DNA, 710 bp, Drosophila melanogaster
  misc_feature (1)...(710): n = A,T,C or G
gatatatatttttgtttattttaaaaaggttgggtattgatttcagtacgtctcccattctagtaaatggttacttttaanagccgttccgatgttattttataggcgatcttcnttgtgcaactcagatcgaaactgaaaaattttacatttccatggtttattttaatgtgaagttaaactgcaaatttctagtctaagcgtagtagttaagattagccttcttcttcgcctgcacttccatgatggcgtccatgaatctcgtgcgtaaccgaattggcggancgacgcagtgcgatcataccacttccacacaaacggtttgcactgggcgccgttgaatcatccgtggatcgggacatcctcgaaatcacatcatgctaactttcatttacgcaatgaattgcataatacggccggcttccccttgggatttgaaancatctacatccnangaccaaccccccaaccgatccaaantccccaatggtcccaatccaaggnaattcnaattcccnctgnggcccactgcntaaggccatccccattnatcttaatccggcgcnttnnctctnaggaaccgnttccatatcctgncnncctccntggttacaaagcccantccccatnccnaaggaatgaccttcgctaccgggtggtccntactntcnccaccnttcttctctncttcgtccacanggctnggcatg

SEQ ID NO: 10 - DNA, 479 bp, Drosophila melanogaster
gatatatatttttgtttattttaaaaaggttgggtattgatttcagtacgtctcccattcagtaaatggttacttttaagagccgttccgatgttattttaaaggcgatcttcaaggtcgaactcagatcgaaactgaaaaattttacatttccatggtttattttaatgtgaagttaaactgcaaatttctagtctaagcgtagtagttaagattagccttcttcttcgcctgcacttccatgatggcgtccatgaagtcttcgtgcgtaaccgaattggcggagcgacgcagtgcgatcataccagcttccacacagacggctttgcactgggcgccgttgaagtcatccgtggatcgggacaattcctcgaaattcacatcattgctaacgttcattttacgcgagtgaatctgcataatacgggcacgggcttcctcgttgggatgtgggaactcgatcttacgatccagacgac

SEQ ID NO: 11 - DNA, 355 bp, Drosophila melanogaster
tggccccaggacgagcgttgcctcgcccgaggacgatataccctgccccataataatcctaaacccataccgaccggcaggtggtcttccagaggagacgataacgacgtagcgtgttcgaaagggacagtggagtcagtggtcggcaaaggtggtcccaggacgagcgtttgcctcgcccgaggacgatacaccctaacccataacatcataatcccagccgggccgactcgtcgtccgtgtcaaggagcaagcaggaccacggaggcaaggcgttgcaggagaaatgccgcaggagcaccgagattgccgaagaagttatccataaggctgtagaataaaatactataataag

SEQ ID NO: 12 - DNA, 171 bp, Drosophila melanogaster
gtatgttaacaaaaattactaaccctataaacattaacgtccatcgggactaaataaagcaaatgtaacacgtctagacattgacataatccctgttcaatatcacgcaattttaaaccatccaacggcagcataaatttcttctccttctcatcctcgtccttacacaca

SEQ ID NO: 13 - DNA, 170 bp, Drosophila melanogaster
tatgttaacaaaaattactaaccctataaacattaacgtccatcgggactaaataaagcaaatgtaacacgtctagacattgacataatccctgttcaatatcacgcaattttaaaccatccaacggcagcataaatttcttctccttctcatcctcgtccttacacaca

SEQ ID NO: 14 - DNA, 162 bp, Drosophila melanogaster
gtatgttaacaaaaattactaaccctataaacattacgtccatcgggactaaataaagcaaatgtaacacgtctagacattgacataaatccctgttcaatatcacgcaattttaaaccatccaacggcagcataaatttcttctccttctcatcctcgtcc

SEQ ID NO: 15 - DNA, 249 bp, Drosophila melanogaster
ddtcaataggwraaaatatagaataataaagatatgaatyaaagattgggtaaagagataggataaamatagtaaaggaagaaagtgtgcatggaattagaaattaggaattaggtttttttdtttttcagataaaaggamaaagaaggaaaaatttaaagaaaggatatggaaaaatgagagaagaaattatagagaaaataatgcatgattgagaatgaagtaagaattgagaggaatwaaattaag

SEQ ID NO: 16 - DNA, 709 bp, Drosophila melanogaster
  misc_feature (1)...(709): n = A,T,C or G
canataatgtctctatcgctgtaataattccancgtaacacgaaggcaatgtgatcagtanatgagaaattttatccatctcttctatttttgcaccagctgccaacaattcnttttttttttcgtctaaaatangaaaatggcttaatagtgacatctcacttgatagcttcagagaaagcaaacgttttcgcagcgccagttgcgacgccaaactttttcgttcataaacggcgtccaaattctcaagaactgacgcgccgtaatgtccttgttgcgaaattaaaaacaatccttaggtacaantcncgaagggcaattctgcanatatccatcacactggcggcgctcgaacatgcactananggccaattcccctatagtgatctattacaatcctggcgtctttacactctgannggaaaccggcntaccaattatcnctgaccatcccttcncacngnttnaacaaagcccngacccccanttgccccgaaggaaggaccctgtacgccntaccgnggtgngtncccntactactcnccnancntctttctttnctnttccctccggtccgtaaccatgggccttgtcatatctcngccancnantatnggagccttggccccaaagntcccaatgatctcnaangactcncganccccccgccntaataggatcangctgnnanacataatcncatcacngg

SEQ ID NO: 17 - DNA, 468 bp, Drosophila melanogaster
cagataatgtctctatcgctgtaataattccatcgtaacacgaaggcaatgtgatcagtagatgagaaattttatccatctcttctatttttgcaccagctgccaacaattcacttagtaagttcgtcaaaaatatgaaaatggcttaatagtgacatctcactcgatagcttcagagaaagcaaacgttttcgcagcgccagttgcgacgccaaactttttcgttcataaacggcgtccaaattctcaagaatctgacgcgccgtaatgtcgcttgttgcgaaatttaaaaacgagtcgcttaggtacgaattcacgaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaactt

SEQ ID NO: 18 - DNA, 416 bp, Drosophila melanogaster
cattgacttggcaaaatgaaacaaaacaaattgaaatctatttgtaatttacatttaagcctaaaaacatatgattatatcaaacacttagttttagtcgataattgtttataatttttcagacacacacacgcaacacacacagacacattcaacttaaagtgcgtaacataaagtaaaataaataaatgaaaacacattaacacgaacaaaacaataatcaagaactggagcggattgggtttcgttttccagcgattacctggagatcaccatggcaaccagtcacactcatttacacttggaatgcatgggagttcttctatcaactaacaaatcctatttcatatacaacacgttaactatgtttgcttggttagttcgctttcctgtcgcttgttataagtacacaatat

SEQ ID NO: 19 - DNA, 286 bp, Drosophila melanogaster
tcaaagcaggtgcaacgttgtacatacatatatagaaagaacaaaatgagagagatcaatctgtaacttgaatgtggttaagtaaagaggtgcatatatatttttttacacgcgtatatagtttgcgtttttcgctttccacacaagatacgtacttcgtagccccccttcccctttccaaatactgtatcacaaagatcataactcaaaatgctattgctttgacttacatcttatttcggtggtgtcaactgcgccaccatacgaaaatacataaattatagcg

SEQ ID NO: 20 - DNA, 706 bp, Drosophila melanogaster
  misc_feature (1)...(706): n = A,T,C or G
atgatacaacaagactttaatgcatgatttgcgcagcttacactacactacaaaaatggaatacatacttttctgcttattggaatagtctacacacttttgctacataggtacaattaagtttgtggcttgccctttgcgaattacaatatggaaacggatacagaacagaaaatagtttaacaataatattgctggaataaacacatccaaggtaatactcagacagcactcgtcatcgccctcatccangatattggcctgctggcgcacatcgatgccctgctgcacaactccgccttcttggcttcggcttgaagncttncccccctcctgttcnggatctcctcantccgtaaaaccctccccncaactctccactccaaatgatttnggcaattcnatcaatccggganaatccatgcccatggctttngtattcccctcccttggcactncnaacccccggntaaacgcattcctgtgttcattcaatccaaggnaatccgcattctcnctgnggcctcactctctaaggccatcccnaatatctntaatccggcgctttaanctatggaacngntacatacctgacatccttccatggtaacaaagcccatccccaatncccangangaccctcgctaccgggttggtcctactatnncacccctnttctcttcttcgtccanatggctngtctttt

SEQ ID NO: 21 - DNA, 459 bp, Drosophila melanogaster
  misc_feature (1)...(459): n = A,T,C or G
atgatacaacaagactttaatgcatgatttgcgcagcttacactacactacaaaaatggaatacatacttttctgcttattggaatagtctacacacttttgctacataggtacaattaagtttgtggcttgccctttgcgaattacaatatggaaacgatacagaacagaaaatagtttaacaataatattgctggaataaacacatccaaggtaatactcagacancacgtcgtcatcgccctcatccaggatattggcctgctggcgcacatcgatgccctgctgcagcaactccgccttcttggcgtcggccttgatgcgtgcctcgcgcttcttgtcctggatcttcttcagtcggtagaactcctcacgctcgagctcgtccagctccganatgatgtaggccaaagtcctatcgattcgggggatgatcacatgctcaatggcgttttggta

SEQ ID NO: 22 - DNA, 483 bp, Drosophila melanogaster
cgggcataaagtaggtgggaaggtaaggaaggtactaagcgcactccaaatctgtttggtaaacattgtagacgaagcatgtggaattaaagccaaacacgataattgtgccgagactcttggccagagattgtcaaggtcgtgcatcttacgcgagtaaatcaaggaaaatgtgagcaggttaaagaaaatttctacctactaaaaacaatattaatgcatctccaaatattagtttcttcctacaggatggtagatggttttggaaatgtatctttttatgtaacctgctctttggtgtcagatccgaattcacgaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcaa

SEQ ID NO: 23 - DNA, 514 bp, Drosophila melanogaster
  misc_feature (1)...(514): n = A,T,C or G
cggctctaatttcattttgtgcatattttggtcctggttctggtgcctatccctcctttttggntcggcccgtgcaggagcttaattaattcccccaaaaatatttataactttgggsccaatacggctgctgttgctgctgctgactactgaracatatttaatttatatttcttggagtgtgtgcggcttgtcaatggctgggaatctaagaaatttatgcatgactgcaacagggtcaagttgcaaagcccttagcctttaatgccatccagctgccgggaaagccgggaaagctganaaaacaaaactgactccttactgaagctgaaactgaaagaacttttagtcctatccagggttgcggatggatccaactccccagataagcagatttatgacctaaacaccgaaactccaatactggaaanacaatcngttttcngtttcgtactggatccgaatcncaaaggcaaatctgcnattcctcaccgcgggcgcycaacatctctaa

SEQ ID NO: 24 - DNA, 430 bp, Drosophila melanogaster
  misc_feature (1)...(430): n = A,T,C or G
cggctctaatttcattttgtgcatattttggtcctggttctggtgcctatccctcctttttggctcggcccgtgcaggagcttaattaattcccccaaaaatatttataactttgggcccaatacggctgctgttgctgctgctgactactgaaacatattttaatttatatttcttggagtgtgtgcggcttgtcaatggctgggaatctaagaaatttatgcatgactgcaacagggtcaagttgcaaagcccttagcctttaatgccatccagctgccgggaaagccgggaaagctgagaaaacaaaactgactcgtactgaagctgaaactgaaagaacttttagtcctattccrggggttncggatggatccaacyccccagataagcagatttatgacctaaacaccgaaactcaaataactgg

SEQ ID NO: 25 - DNA, 213 bp, Drosophila melanogaster
aacattttagattgaaacacattccaaaagtctaagactctagcttcacaacggtcgtcttctcggacacgtacagbbcgtcaaggaacttacggatatccttgttcttgacgstcgtggactgctggatgagggcggcagatccggagacagactcaatatcgttccgtamscgtaaggtyggccctctggavagtgaggtcacccaccgcg

SEQ ID NO: 26 - DNA, 365 bp, Drosophila melanogaster
aacattttagattgaaacacattccaaaagtctaagactctagcttcacaacggtcgtcttctcggacacgtacagaccgtcaaggaacttacggatatccttgttcttgacggtcgtggactgctggatgagggcggcagatccggagacagactcaatatcgtttccctccacgataagttcgtccttctgggcagtggagttgaccacggtgacgccaggagccatctccacacgacggatgtacttctcacccaagaagttacggatctcaatgaccgtgttgttctcggaggtgacacagttgatggggaaatgggcgtacacagcacgcatcttgtactggatccgaattcacaaaggg

SEQ ID NO: 27 - DNA, 212 bp, Drosophila melanogaster
acattttagattgaaacacattccaaaagtctaagactctagcttcacaacggtcgtcttctcggacacgtacagbbcgtcaaggaacttacggatatccttgttcttgacgstcgtggactgctggatgagggcggcagatccggagacagactcaatatcgttccgtamscgtaaggtyggccctctggavagtgaggtcacccaccgcg

SEQ ID NO: 28 - DNA, 691 bp, Drosophila melanogaster
  misc_feature (1)...(691): n = A,T,C or G
atgatacaacaagactttaatgcatgatttgcgcagcttacactacactacaaaaatggaatacatacttttctgcttattggaatagtctacacacttttgctacataggtacaattaagtttgtggcttgccctttgcgaattacaatatggaaacggatacagaacagaaaatagtttaacaataatattgctggaataaacacatccaaggtaatactcagacagcactcgtcatcgccctcatccangatattggcctgctggcgcacatcgatgccctgctgcacaactccgccttcttggcttcggcttgaagncttncccccctcctgttcnggatctcctcantccgtaaaaccctccccncaactctccactccaaatgatttnggcaattcnatcaatccggganaatccatgcccatggctttngtattcccctcccttggcactncnaacccccggntaaacgcattcctgtgttcattcaatccaaggnaatccgcattctcnctgnggcctcactctctaaggccatcccnaatatctntaatccggcgctttaanctatggaacngntacatacctgacatccttccatggtaacaaagcccatccccaatncccangangaccctcgctaccgggttggtcctactatnncacccctnttctcttcttcgtcca

SEQ ID NO: 29 - DNA, 677 bp, Drosophila melanogaster
  misc_feature (1)...(677): n = A,T,C or G
cgggcataaagtaggtgggaaggtaaggaaggtactaagcgcactccaaatctgtttggtaaacattgtanacnaagcatgtggaattaaagccaaacacnatttttntgccnatactcttggccagagattgtcaaggtcgtgcatcttacgcgagtaaatcaaggaaaatgtgagcangttaaagaaaatttctacctactaaaaacaatattaatgcatctccaaatattagtttcttcctacaggatggtagatggttttggaaatgtatctttttatgtacctgctctttggtgtcanatccnaatcncgaagggcaattctgcaaatatccacacctggcgggccgctcgaacatcntctaaanggccaatccnccnattatgaatcctatanaatcnctggccgtcttttacactctgangggaaaccnggcnttnccactaaccctgcacctcccttccnctgnttatacaaaagccncatccctccacattgcccctaagnatgaccccttcgcctanccggggtntgttccntactcttcnnctaccccctcttcctcttccnttcggtccnactaagggcctggcattttgcccccaataaggngncttgcccnaagtcccaatgtctcnangactccgaaccccncccctaaaggacncctgaaa

SEQ ID NO: 30 - DNA, 141 bp, Drosophila melanogaster
atgatataatggattggtaatcaattggcatcgaaattaatttacgatataaacaccacttaacgccgcctcaacctaattactgtctgcatatgcaatagaaaacgtatataaattaattaaataaaaaaaaaggaaagt

SEQ ID NO: 31 - DNA, 322 bp, Drosophila melanogaster
  misc_feature (1)...(322): n = A,T,C or G
atttcgcgacaggcttcggcacgccagtatataacccaaaacacacnaacntcaggggctggancgcgtcactgccgtgctcctccagccggcacagtcattccccgcccccacaccaancaaaaccggccgcttgtgcanatgacataggcgcgaccanccaactgacccggctgaccagacttgcaccgtgcgccatcaactggaatcttggccacaagcacagctttagtttggcccgctatcccncacacaaacccagantgggggtctatggaagaccacaagtngttgcgttggaactgctaaanatttnnactgt

SEQ ID NO: 32 - DNA, 308 bp, Drosophila melanogaster
  misc_feature (1)...(308): n = A,T,C or G
acgcatacaatatatgattatacatacatatatatatttacaatgataaagaatgtaaggcccaagccaagcaaacacatatgtaacgtgtatttgaaccacgtacttattatttacatgtttacatatacgaacatccaaagcaaaggtatatacacgtataggactcaacatttacaaattcaatattcttatatgtggaaagcanagcgttacgattatctcccanctaactggaagcgattgaatgtctatacatnatttgtaatgccaaataaaataaaatatatcacgttatattaaacagt

SEQ ID NO: 33 - DNA, 201 bp, Drosophila melanogaster
acgcatacaatatatgattatacatacatatatatatttacaatgataaagaatgtaaggcccaagccaagcaaacacatatgtaacgtgtatttgaaccacgtacttatatatttacatgtttacatatacgaacatccaaagcaaaggtatatacacgtataggactcaacatttacaaattcaatattcttatatgtg

SEQ ID NO: 34 - DNA, 187 bp, Drosophila melanogaster
acgcatacaatatatgattatacatacatatatatatttacaatgataaagaatgtaaggcccaagccaagcaaacacatatgtaacgcgtatttgaaccacgtacttatatatttacatgtttacatatacgaacatccaaagcaaaggtatatacacgtataggactcaacatttacaaattcat

SEQ ID NO: 35 - DNA, 687 bp, Drosophila melanogaster
  misc_feature (1)...(687): n = A,T,C or G
agaattaccacgcgaacacaattctgttttattgtttttaatacatatttaatctttgcganaagagctagtgtaggtagtctggaatttttcatatatttaacgatatccattggtaatgattacatagttggattagaactaatacttgtagcagttaatggaatgttcaccaccgctctggatcatcgttgctggtcagctggcaaggcatcatcacgcacttttccatgcggacgcnatccttgcacttgtggctcaatcggtgttcattaaggttcgggttcgttggcgaacggcattatcgccacacgttgcggtgcatggtgtccaagcggaacactcccaattancnacactcgtcctgcggtccggttgcngactcttaccacatccttcctctccaatccccgtccctgattgattacnntcatccacccctggtaacacnattccaacttccagttgcttggaaatgctgcnccctactccgaatacgacnctcccttcccatgaaccnccccagagcttgcacgtggaccntcatcatccaagnaatctgcattctcccgcggcncactcttaagccatccccaatatcttaatccgcccttaatctatgaaacgnttccatacctgncancctccctggtaaaaancccatctcccttnccnangangaccctc

SEQ ID NO: 36 - DNA, 311 bp, Drosophila melanogaster
  misc_feature (1)...(311): n = A,T,C or G
tcccatcaattcgttactcatcaattgaaatttcagatttggtaatgctaaagggctatcatgattgcagttctatgaagtggatcaaagcgatttcgggtcaaagattgcgggtcgctgctagaaagattgatctctagtgcttctccagtgcttgcttagttcggcgagggcataaccttgatgcgctccaaggcttgtttctccanggtctcgcggtgcttgggatcggcgatctggataagttcgtacatcctctggcgcacattcttgccgaacagcgaagcgattccatgctccgtgacgactta

SEQ ID NO: 37 - DNA, 670 bp, Drosophila melanogaster
  misc_feature (1)...(670): n = A,T,C or G
cccatcaattcgttactcatcaattgaaatttcagatttggtaatgctaaagggctatcatgattgcagttctatgaagtggatcaaagcgatttcgggtcaaanattgcgggtcgctgctagtaaaatagtgatctctagtgcttcttcagtgcttgcttagttcggcgagggcataaccttgatgcgctcgaagcttgtttctccagggtctcgcggtgcttgggatcggcgatctggataagttcgtacatcctctggngcacattcttgccgaacacgaagcgattccntgctccgtgacnacttantggacttnggcacgcgaanttgacaacccagcgcctgccttcacgttnggaacaatcttgctctccccttgttggtggtcaatgcatggcnataattgcacacccatccatcnaaacctccncgtccccnaatnaattcacctntccccnaaccgggattaaanccggaacatcatctacncctgtcntccattccaatccaagggaatctnnattcaccngcgggcnccaacatctcnaaggccatcccaatatntttanatccggctcttaactctatggaacnncttncataacctgantccttccctgtttcaagccncatccccncttcccaagataccctcgctaacgggtng

SEQ ID NO: 38 - DNA, 192 bp, Drosophila melanogaster
accatttaattattaaatatgatttatttatattaatatgtagtcaaaaactccgtgttagctttaatttacctaccccactttggatctaaataaatatgttaaatgttgattcaagcgtgataatttatttggaacagcattgcgaaaattgrgtagtycataatgttttttcttcctggkcactgagca

SEQ ID NO: 39 - DNA, 362 bp, Drosophila melanogaster
  misc_feature (1)...(362): n = A,T,C or G
gctgaactggacctgaatataaacntatacacatctattgcaacaangatacacaccttgctgttaaccacctgcaacatccaancttcttacatccctggtgttagttcgacanactctacatttccccacctctgccgantgctganagttaantcatgggaacagganatnccncttccccaaagggaatattttntgttnaaataaatactgcctcttgcngttcaacgtananananaaataccnaattccgaaaggggccnaanttnccgggcncanannggcctgcctcntagggaatcnccanccccttnttatangccctcttccgcctataaacttgtgccngaanccccng

SEQ ID NO: 40 - DNA, 322 bp, Drosophila melanogaster
  misc_feature (1)...(322): n = A,T,C or G
atttcncgacaggcttcggcacgccagtatataacccaaaacacacaaacgtcaggggctggaacgcgtcactgccgtgctcctccagccggcacagtcattccccgcccccacaccaagcaaaaccggccgcttgtgcagatgacataggcgcgaccagccaactgacccggctgaccanacttgcaccgtgcgccatcaactggaatcttggccacaagcacagcaatagtttggcccgctatccccacacanaaacccacantgggggtcnatggaagaacacaagtggttgcgtggaactgctaaaaatataaaactg

SEQ ID NO: 41 - DNA, 323 bp, Drosophila melanogaster
  misc_feature (1)...(323): n = A,T,C or G
atttcgcgacaggcttcggcacgccagtatataacccanaacacacaaacntcaggggctggaacgcgtcactgccgtgctcctccagccggcacagtcattccccgcccccacaccaagcaaaaccggccgcttgtgcagatgacataggcgcgaccagccaactgacccggctgaccagacttgcaccgtgcgccatcaactggaatcttggccacaagcacagcaatagtttggcccgctatccccacacagaaacccagantgggggtctatggaagacnacaagtggttgcgtggaactgctaaaaatataaaactgt

SEQ ID NO: 42 - DNA, 176 bp, Drosophila melanogaster
caagtgcggcggcgacaagaaatccgcctgcggctgctccaagtgagctttcccccaaaaaagatctggagtagaggcgctgcatcttgtctccgaactgatttctgtataactcccaatactaaaacgacatgttttctcatttacacaccctgcaataaatgtccaattaaagt

SEQ ID NO: 43 - DNA, 323 bp, Drosophila melanogaster
  misc_feature (1)...(323): n = A,T,C or G
atttcgcgacaggcttcggcacgccagtatataacccaaaacacacaaacgtcaggggctggaacgcgtcactgccgtgctcctccagccggcacagtcattccccgcccccacaccaagcaaaaccggccgcttgtgcagatgacataggcgcgaccagccaactgacccggctgaccagacttgcaccgtgcgccatcaactggaatcttggccacaagcacagcaatagtttggcccgctatccccacacagaaacccacantgggggcctatggaagaccacaagtggttgcgtggaactgctaaaaatataaaactgc

SEQ ID NO: 44 - DNA, 176 bp, Drosophila melanogaster; identical to SEQ ID NO: 42

SEQ ID NO: 45 - DNA, 323 bp, Drosophila melanogaster
  misc_feature (1)...(323): n = A,T,C or G
atttcgcgacaggcttcggcacgccantatatancccaaaacacacaaacgtcaggggctggaacgcgtcactgccgtnctcctccanccggcacngtcnttccccgcccccacaccaagcanaaccggccgttgtgcagatgacataagcgcgaccanccaactgacccggctgaccagacttgcaccgtgcgccatcaactggaatcttggccacaagcacagcantagtttggcccgctatccccacacatanaacccagattgggggvvtatngaanaacacaagtggttgcgtggaactgctaaanatatnaaactgc

SEQ ID NO: 46 - DNA, 362 bp, Drosophila melanogaster; identical to SEQ ID NO: 39
  misc_feature (1)...(362): n = A,T,C or G

SEQ ID NO: 47 - DNA, 416 bp, Drosophila melanogaster
agtttacatgtactttattcgttttgtatatcccagacagatagagttatttattgaacacttcaactggctaggtcgtattagggtctgcttgtaacttttgtgtcagtaaccactctaaaatagtataatgctagtaattctacccatcaacccattgtatacatacttatattcaaaaccctttcaccacatttctaagcctagattatggataatgcctctaatatgtaacgagtgcttaggtcaccttagccagccgctggtcgatgcatttctggctgcgaaggtcgaaccaatttcccggactgcagtaatgcaaaaccgcttttcccttcaagcaaacataatacttgttatgctgcttgacgtctccaaatcgtgtatcctctttcactttggtgcaatcgggtacc

SEQ ID NO: 48 - DNA, 413 bp, Drosophila melanogaster
caaatagtttacatgtactttattcgttttgtatatcccagacagatagagttatttattgaacacttcaactggctaggtcgtattagagtctgcttgtaacttttgtgtcagtaaccactctaaaatagtataatgctagtaattctacccatcaacccattgtatacatacttatattcaaaaccctttcaccacatttctaagcctagattatggataatgcctctaatatgtaacgagtgcttaggtcaccttagccagccgctggtcaatgcatttctggctgcgaaggtcgaaccaatttcccggactgcagtaatgcaaaaccgcttttcccttcaagcaaacataatacttgttatgctgcttgacgtctccaaatcgtgtatcctctttcactttggtgcaat

SEQ ID NO: 49 - DNA, 885 bp, Drosophila melanogaster
  misc_feature (1)...(885): n = A,T,C or G
rtnstartmnctmrtnstttctamcmmntdskasamdsdystrmrdtacastanyrmrmachndsnnnngnagatacgccaagctatttaggtgacactatagaatactcaagctatgcatcaagcttggtaccgagctcggatccactagtaacggccgccagtgtgctggaattcgcccttcgtgaattcggatctgactgcaagtgcggcggcgacaagaaatccgcctgcggctgctccaagtgagctttcccccaaaaaagatctggagtagaggcgctgcatcttgtctccgaactgatttctgtataactcccaatactaaaacgacatgttttctcatttacacaccctgcaataaatgtccaattaaagtaaaaaaaaacaaaaaaaaaaaaccgaattccgaagggcgaattctgcagatatccatcacactgggggccgctcgagcatgcatctagaaggcccaattcgccctatagtgattcgtattacaattcactggccgtcgttttacaacgtcgtgactgggaaaacctgggtttacccaacttaatcgccttgcacacatcccctttcgccagctggcntntacaaaaaggcccncgattgccttcccacantgccacctgaatgggaatgaaccccccgtaccggccttaaccgnggttggtggttacccacntacgcaacntgcacccctacccnncttccttttcctcttcccnttccggttccctcacctantggggcctaggtcaatttcttnngccaccaaatntagtangtctttgcccccaaaagttccctaattgatcttctaaatganntcnngaaaccncaccgtntttantaaggatgcatcgcnngtaaacatcc

SEQ ID NO: 50 - DNA, 496 bp, Drosophila melanogaster
cttgatccagcaatctatttttcacaaacgccaatgtcaaattttcttcagataatgtctctatcgctgtaataattccatcgtaacacgaaggcaatgtgatcagtagatgagaaattttatccatctcttctatttttgcaccagctgccaacaattcacttataagttcgtcaaaaatatgaaaatggcttaatagtgacatctcactcgatagcttcagagaaagcaaacgttttcgcagcgccagttgcgacgccaaactttttcgttcataaacggcgtccaaattctcaagaatctgacgcgccgtaatgtcgcttgttgcgaaatttaaaaacgagtcgcttaggtacgaattcacgaagccgaattctgcagatatccatcacactggcggccgctcgagcatgcatctagagggcccaattcgccctatagtgagtcgtattacaattcactggccgtcgttttacaacgtcgtgactgggaaaac

SEQ ID NO: 51 - DNA, 936 bp, Drosophila melanogaster
  misc_feature (1)...(936): n = A,T,C or G
acatcaatgctagtgcttccttttaccgaaaacctattgaatacgctaaaaaattggaatagtcgcaagcggaagtcggccaaaaaaatccttaagaattttggaaccagttcttctacttgtcgtatcgaaccaggcgcgtgtcgtcgccgacctcctccagatcctttggatcgcggcggaagcgataagtgcccacatcctggttggccgattccggcaacgtcaccttgatgcccttgtactcggttcgaccttccctgacctccggcacccgcagctccatctcggccttgtactcgtcatcgttaccaatgtccacgtcctggaccgttcttttgcacggtgggatcctcctcgtcctggttccagccatcaaatctcgatggggacaatggggttgccgtcgacgcctacgacggnactangtgcgccantagggcaggatctccacgggtaatctccagaaaatcggaattctctggctgggttggcagactcaaactgcantcccgcantccacnaatgtttgggtcanctccntttgaaatgggaggtatgggtccatcaaggnagcgaaattcacnaaanggggnaattctgcannatatccatcacactggngggccgctccaagcaatgcatctaaaagggccccaattcctccctaatangngagtccgtattacaaattcaacngggccgtcgttttanaanngtcgggaatggggaaaaacccngggngntaancaaacttaatccnccttggaagcanaatcccccttttcgcaagangggngtatnannaaanagggccgcaacgantgncccttcccaanaantttccnancctgaatngngaatggacncnccctgtnnnggggcaatnaacccggnggggttgntggtanccncaangtntacggctaaanttgc

SEQ ID NO: 52 - DNA, 629 bp, Drosophila melanogaster
  misc_feature (1)...(629): n = A,T,C or G
gtttgcaaaccttcctatttaagtaaagtgtttgactctggctcccaaagcttnccttgggaaacgggaaaaattctctacantgtatatgtgcgcatgcaaactcatttggtaaattacacatnaataaatatgtataacaacaactanacatatgtnnatggaaaataaaaattttcagtaacgactnaactcgantgtcggtagcatnaagggannaagtcgtcnantgttattatctaatttgcagcctgtattgtccagatacaatatgtnatngatgcantgtatatctnttgtgtacatanatatatgtttaaggcgactcctatttntctgcntgtgcatatcgatcaaatgcctactttcntgattgtttngtgtgtttccnctaaggaaaanatacatgtgttatatcnynaaaagaattgtatcgtattaggtttgcttcctcaaacatccaccaaaaatcgntntcntntananccnaaaaatacgaaaatnnttgtgccttaaaaanaaacaatcgaggnaatcccaantccnaatgcggngtcactcngntaccatatgctcnaancttccctggttcaaagcccatncccacttncccatgangaccttcgctg

SEQ ID NO: 53 - DNA, 977 bp, Drosophila melanogaster
  misc_feature (1)...(977): n = A,T,C or G
cgtttgttgccggtattgttggttggtaggttgtttgttagtagagagagagagaaccggtacgctataaaactacgctcccattgccggattgttattggagaattgcgcccgccacccaagcagccacccacgtatcacccgctcacaagagcggaaaatggatacagtccgggttcctggcggtagaaccgtaatttctgtgatttgctttttttgtgttaagtaagtatttaataagtagattactgangtttgctgctccgcgggcgattcccttaggcggccacttcgctangcctcggncccattctgaacctcatcctttgtgctgggcctcatcaagcancgaattcacnaagggcgaattctgcagatatccatcacactggcggccgctcgagcatgcatccgagagggcccaattccgcccctaatagntgantccctattacaattcactgggccggtcgtttttaanaaccggtcnntgactggggaaaaccctgggcggttncccaaacttaattcnccttgcaagcacantcncccctttcgccaagctgggngtaattancgaaaagnaggcccgcacccgatnggcccttcccaacnngttgcgcaggccngaaannggccganatggancgcgccccggtnagccggngcaattaatccgcnggnggggtgttggtgnggttaanccgcaaaccgtgaccggcntataccttgccaagggcccctanctgaccnggntcnttttcggctttcnttcnccttccttttnctnggcnaaanttcgnncgggttttcnccggtcaaagctcntaaatnnggggggntcccttttagggnttccnnatttnaggggctttnacgggnaanctccaanccccaaaaaancttgcttnnnggtgaangggtnnacgttnntggggcancncccctnataaagggntttnccnctttgnagatgt

SEQ ID NO: 54 - DNA, 875 bp, Drosophila melanogaster
  misc_feature (1)...(875): n = A,T,C or G
gcgatcttacaaaataaataacagcaaatagaaagataaacttacatataagcgcaatattcaaatgtttagtggcgtctacgaaatgtttttcaattactgctggtgtaagacacatagataataaatgtgatgtgttttgtgtgtttttttangtttggcctaccagaagtgtgctctaaatatataccaatgtgaatcgaaatcgtagctccttgcgttctcctatatacatgtgcaccgtgagatccatagtcccatcgttttcggtttaagttacccycgggcyyyggcagattcgnaatcatatgcacgtataaagatagactgcgtgcacagctccggccctcctcctgggaaaacgcatagccataccgaattatccgatcccaangcatacatgggtagaangatctcgggtccgttcatcaacttcggganatgtcgcnncgntccggtctccgtttccgcgaacagccttccggtcagtgtcctannncacgggtattaaggtaccaagtttgcaagatcacatcgatcagcagcgtgggtaaatgngggcaccagcagtcaaggcangcgaattccaccnaangggcgaaattccggcaagaataatccatcacactggggggccggctcgaagcatgcaatcctagaaggggcccaaaattccgcccnatattgaggtccatattanaaaagttcaatgggccgtccgntttannaaacgttcntgantgggaaaaancccnggcgtttacccaacttaaatcncccttncaagnaaatnccccttttcagcnaanctgggcgtaatnnncnaaanangncccgcaccggntgcccctttcccaacaatttngcccaagnct

SEQ ID NO: 55 - DNA, 465 bp, Drosophila melanogaster
  misc_feature (1)...(465): n = A,T,C or G
ggggtcgtactcggtgaggaaatccaagcgcttatcatgcttcactttgcagacaatcagtacatcgattgatgaggaaaaagaagaccccttgaatgggtcgataatcattactgtccaactcgattagagctccctcgttgaggaaggtcttgccttccagattgccattgaagccctggaccatttccttgaccgcccgcgtggcatggctattctccagatcctccgtcgccgtantgctctccgcctccaaactctctgccttcaggtgactggaagtcttgccatccgtcatggtggccanaatattgcgctgctcaatcagaatgtgcgacagttgatacatttccgactcgagatgtgatatctccttggncgtctgtataaactccatatagttctttttgcatgtttgcttgagcgttgctgccgtcgtttcgttgtaggcctcgatttcctttt

SEQ ID NO: 56 - DNA, 238 bp, Drosophila melanogaster
  misc_feature (1)...(238): n = A,T,C or G
tgctgcctgctccttttgggactcctgggcttcctagctgctcccggcgtcgcctcgccatctcgccacactggaccaggaaacggatcgggatctggagctgggtccggaaatccgttcaggtctccaagctcacagcaacgaccactgtactacgacgctccgattgggaaaccatcnaagactatgtacgcctgacgtanagaatgaaacaanaaagatttgaaacncctanact

SEQ ID NO: 57 - DNA, 237 bp, Drosophila melanogaster
  misc_feature (1)...(237): n = A,T,C or G
gctgcctgctccttttgggactcctgggcttcctanctgctcccggcgtcgcctcgccatctcgccacactggaccaggaaacggatcgggatctggagctgggtccggaaatccgttcangtctccaagctcacagcaacnaccactgtactacgacgctccgattgggaaaccatcgaagactatgtacgcctgacgtaaagaatgaaacaataaagatttgaaacgcctaaact

SEQ ID NO: 58 - DNA, 238 bp, Drosophila melanogaster
tgctgcctgctccttttgggactcctgggcttcctagctgctcccggcgtcgcctcgccatctcgccacactggaccaggaaacggatcgggatctggagctgggtccggaaatccgttcaggtctccaagctcacagcaatgaccactgtactacgacgctccgattgggaaaccatcgaagactatgtacgcctgacgtaaagaatgaaacaataaagatttgaaacgcctaaact

SEQ ID NO: 59 - DNA, 253 bp, Drosophila melanogaster
attacgtccctgccctttgtacacaccgcccgtcgctactaccgattgaattatttagtgaggtctccggacgtgatcactgtgacgccttgcgtgttacggttgtttcgcaaaagttgaccgaacttgattatttagaggaagtaaaagtcgtaacaaggtttccgtaggtgaacctgcggaaggatcattattgtataatatccttaccgttaataaacatttgtaattatacaaataaaaacaatttacc

SEQ ID NO: 60 - DNA, 236 bp, Drosophila melanogaster
  misc_feature (1)...(236): n = A,T,C or G
aacaggcaaaagcgatatcagtaataaactaaacgcaccaattgtttaaataaccaaagcgttaagaaaaaaatcaaagacaaagccacggcaaaaggcgcagacaacaagttgtttgcttttagttcgcgttctccttattttattttccttccgttcgattttccacgcacgcgcgtcgcagaaacgtcaaattgaaaacatcancagttgaaagccaactgttgcattctacc

SEQ ID NO: 61 - DNA, 247 bp, Drosophila melanogaster
  misc_feature (1)...(247): n = A,T,C or G
ttcaggcatcttccttctaattctggctgtgggtttggcacaaatgccgctgcaggtggccgcccagggccaaaatggacattcgcagggacagccgccaagaccgccaaatggcaatggaaacggcaaccanncagagtggacaaggacaaagcgggcagaacaactagaactgggatatttctggagggggacaacacacctcctcgccactttcccagttacttaaataaacactttccccagc

SEQ ID NO: 62 - DNA, 767 bp, Drosophila melanogaster
  misc_feature (1)...(767): n = A,T,C or G
ctaattgcgctccatccatttgttcctgtccggtgattcccacatctttaatggtggagttatagaaattattttgaataatcaaatcatctccaattatcttcactatttcactcaaagacatggtttttagcgtgctggtcgtgttgcttccaattgcgctgacggctttcgaccatgatccgaattcacnaagggcgaattctgcagatatccatcacactggcggccgctcgancatgcatctaaaagggccccattcgccctatantgagtcctattacaattcactggccgtcgttttacaacgtccttgaactgggaaaaccctggccgttaccccaacttnatcgcctttgcagcacatccccccttttccgccagctnggngttaataccaanaaggccccctawtawtgacactatagaatactcaagctatgcatcaagctwrrataccgagcawcggatccamataagataancagagaccagcacaagtwgtagcatrggabayatatacagcccatatacggagamayatatcaggatatwtwtatatatatatatataaacagaaacatacatatwtatacagtatatawgcamaaaaaaatacattatataaaaaaatatatacragtatatamacacacacvagtatatatatatacgtacgarcacgtacgcatwarcacacacacrvcacggacacacaatwtacrcgacgcacgcacatttahacacaattahtatacacmtaccaa

SEQ ID NO: 63 - DNA, 353 bp, Drosophila melanogaster
  misc_feature (1)...(353): n = A,T,C or G
tawtgacactatagaatactcaagctatgcatcaagctwrrataccgagcawcggatccamataagataancagagaccagcacaagtwgtagcatrggabayatatacagcccatatacggagamayatatcaggatatwtwtatatatatatatataaacagaaacatacatatwtatacagtatatawgcamaaaaaaatacattatataaaaaaatatatacragtatatamacacacacvagtatatatatatacgtacgarcacgtacgcatwarcacacacacrvcacggacacacaatwtacrcgacgcacgcacatttahacacaattahtatacacmtaccaa

SEQ ID NO: 64 - DNA, 609 bp, Drosophila melanogaster
aatttttagcaatttcttatttggtttttcggtactttctctagctgcttttacttgatcgcacatatatatatatatatatattctatacatatacatattcatatgaatatatcttttatcatctttaagaggagattttcagtgtctgtgtgggtgtgtgtgtttgtgtatgcttgtatgtgtccggttgtcctatagccatttgaaccactaagaatttgtagccggggaagttgctatcaaatagagttgctcaacaacggctctggctcgggttgaaggaatttttggaggtcgaggggagccaacgacacaacgcaagctgccccaaaaaaacgggctaagaaatcaggttgggctaatgaaatacaaagcttgcaagggcaagaagaagaagaagactgagcactttcttttcggctgcatcgcttacaaccagttcatagtgcgcctctctccgcgcttctcatcgatggtaggtaagcccttgtttcaaatgatgtgaatgggtctaattaggagtttgtctgtctgtgtctgtattgtgtctgcacaagccagagaaagagaggctggggagaatgggagaaagtgggtgatgggagg

SEQ ID NO: 65 - DNA, 554 bp, Drosophila melanogaster
taaacaaaagaaaaacaaaattccttttgaaaatgcaacattaacaaatagaaagaaacaaaacagaacaaacacgtaaagaaagaggccactacaaaactgaaaagaaaatgtgaaaaatacaaaatttcgtttagccattaagattgttaagaatcagagtgttagatgtagatgagcaagtgaattttgtagggctttgctaccagttttacctgcttaatgaataagggtaaaacattcatatgattggattggaagaatatatcgggaatgctaaaaattattggagtataagttaaatacaactgcgatttatctgtttaagttttaaatgctatattaacgatgtataactttggttcaatgttttagtcataggtttttacatttaactcaatgtggggagagagcttttaaatagatcatacgaacctacatattacatttatcggttattataattgttttggccctctcatccaatatatacatattttatggtcctaggttgtcttttttaagttttccattttgttaaagaaagttcgatt

SEQ ID NO: 66 - DNA, 647 bp, Drosophila melanogaster
  misc_feature (1)...(647): n = A,T,C or G
tggactgatatgcaaaaaagcatttcaccacggcacctgcgcatataatggtggatagcctgtggaacgtccttatcttatcgtgtaaggtggacacgacacgaacactaatcagagaatagagcagttctaactcacaatattgataaacaaagtaagggccagccgagagatacacgcgcatttattggcagcaaacagaagccaaaactacggacatgtccgaatcgggaatcaaaaagttgagccaggagcggactcgcgaatggttggctagtcaggaggacgaggaactggagtccattgcagagtcctcggttgtggacagcttggactacgattataccgaggaagaggaggatgccgaccaaaataccagtgaagaaatcagcactatgacactaggcactcaaatcgctaccaaaaagcattcgatcatcagcgacaccataagggaccttatgaactcgatcaacagcattcagactttgggcaacgttaatataagcaactccacgaacgtccatatcggcaatgttaccaatattaatggaaatatacaaatcatagccgatggccttactcaaaaccgaagagatcggcggcatgtttcaccaccgagagataacgcttccaaaactccgacn

SEQ ID NO: 67 - DNA, 600 bp, Drosophila melanogaster
gttttcaaacgctcagcggagaaaatgtaacggacgaacgcggctggcaaaactcacagacggtacaagagaaccagaataaaaaaggactccacaagaaacggcaactcgacaaaatctatacaaaagtgtctggctcgactgtgtgtgtgcttctgagtgaatgcttgtgtatgtgtgtataaattagtttggttgtgtgagttgttagagtcaaagaactaaaataagactttcagatctagcaaatatgtcccatagttccccgagacgcgtatccactgctgtagccacttaacaaacaatgcccaaagttaaggcgcacggaatctctaataatcgaaaccaataaaatgagccccgttgcctgcagcaccaacactaacatcggtcacatcgagcaggttgcaggcaatcaaaggacaaatatagctgggataagatcaatccaaattggaacaaccacaatcacaacgatattgaaccagcgatgagatggagcgtccgttgggatgacgaactcagaaactcagtaagggagctgcaactgatactgaaactgaaacagaaaccacagcggcactcggaatttagaggcga

SEQ ID NO: 68 - DNA, 598 bp, Drosophila melanogaster
ccgccgagcgcctgctgcagcatcccttcgtccagtgcgagatgtccttgcgggtggccaaggagctgctgcagaagtaccagagtcccaacccgcagttctactactatctcgatggcgatgaggagtctgtggcaggagtgccacaacgcattgccagcaaaatgacgtcacgcaccaatggcgtgccagcgcaaaatcacacactaaaaacaggcatgacgacgaactccacgtggaatgagcgatcttctagtcccgaaacgttacccagtgacatgagcctcttacaatatattgatgaggagctgaagctaagagcgaccttgccactgaacaacgacaccaaagatccactcggcgccgagtgcagctgctcctcccacaatggaggagccgccggaggaggaggaggaggaggagttggagtaggagcaggcggagcagccgcgagcggcagcagcagcagcagcggaggcgcaacagtcggcaccactcatcatcagcaccaacagcaccaccaggatcaccaccatccgaatcatctgcatcagcatcaggcccatcaattgccgcaacagcagcagcagcagtcaca

SEQ ID NO: 69 - DNA, 420 bp, Drosophila melanogaster
cagctggacgcgccgagcatcatggacgccttcctggacaccgagcgacagagaatcgagcgcgagcagcaattggcggcggcggagcaggatgccgatcgccgggcggagcagaaccggctggaactgtaccagattttggccgcctccgagcctgatccgcaaccttaccagaggaagccggcggcacagccgaatgctatggaccaactggaggccattgtggagcagcagcagcagcgcgagctgaaggagcagcaggagcaggccaaggcaccggtctacgtgcctcccgaggaggtgaacgagtcgagcgagctgtacttcccggacaactttgctcctttcaagagagcaaggggtcgctccaggggaggattggccgaggaggtggaggactaacagccgaagcgctccttc

SEQ ID NO: 70 - DNA, 547 bp, Drosophila melanogaster
aagcgtgccagaaatggcaacgacagttcgggttcggactcgaattccagcagtccgcgccagcaaggcagccctccagtgatctgtgaggatgcggctgcttgcgcagctctctccggttacactgtggatcagctctcggatctggccagtcactgcccagtgctgagtaacaacaatgctgtgggacctaccggagttagtggtggtggcgatgcggataccaacaatgtgaacaccactccccgtcagtgccctcttcgcttggtgggcggtcaggaagtgatgggccagtgcccagtgccgcacaatcaggcaatggttcctgccaaatgtccagtagcgcatgcagactctggggattccttcagcgccaagagtggaagtggaggggaatcggccaccactgctcactgtccactacagatgcccgtgggacaggacttcatgggcgaatgtccgtacgttaacaacgatgtgaaggtatcctttgcccaagctggaaagtgtccagtgactggcggtgtggcaggagcatcagcttcta

SEQ ID NO: 71 - DNA, 605 bp, Drosophila melanogaster
atgaatcctctggacaaaatacacgctctagatgagatcgaaaaggagataatcctgtgcatgcaaagtgcaggacaagccttgcaggagttgggcaaggaaaagtcttcccagaaaaatgcggagacccagtcgcagcagtttctcaagagtctgtccagcgtggaatcgaagctgtccgagcagatcaactacttgacccaggtgtccacgggtcagccacacgagggttccggctatgcatccgccaaagtgctccaaatggcttggcatcgcattcagcacgctaggtccagagtgcgtgaacttgaggaaactaaggccaaacactcacatgcagctcgtcagcagttgaagcgtcacaggaacatgccgccgcccagcaacagcagcagcaacaacaacagcagcagcagcaacaacaacagatgcaacaggcggcacaacagcagcaacaacaaaccggaggaggaaatgccggcagcggagatcatccctgggcggagactcctcaatgtcaaccaactaatcttgcgctatctttaagggtaagggttttaaattttttagagtgcattccgaaaaggcacattttgtccaccaat

SEQ ID NO: 72 - DNA, 630 bp, Drosophila melanogaster
tagatccgacagcacagtcatgaaatcagaccgagaagccggtcgtgccgattcgcgatcctggcgggtccattgctcgtcctcgtgcaatcggacattgtattcctcctgattctcatttccatcgggtcgcgaccagatgagcttcaatccattgccaataagcacaatatcgtggccacgctcatagttgccatatgactccactattagactgtacgacaggcggccaccgtacgagaatagctggttgcccagcacacttcccctaagactccagtacttgggcagataggaggtgtgcgtgtaggtatacatattcctagatatgtcgggaattaagttctcggtgtcctggacagctccgctttcgtctgtaattaatggtgcgttaagaataaagtccaccggtattagctggcggtacagagctgccgaacgacactggctggccaatccagagcagtagcactctttgcagccatcctgattttgagcagacagtccataggttccagggcggcattggtcgcattgatcaccaatcacgtttctcttgcacaggcattcgttgccgcggcaatcatagatgccctctatttggcaataggccgtgcattccaaagtttg

SEQ ID NO: 73 - DNA, 638 bp, Drosophila melanogaster
taaagacccgcattgctgaagtgatgcgcgatgatattggttatggaaagaatcggactgtcgaggtgcgaacagaggatgaagtaaccgccgatatggtggcacattcgcatgccgccgtccatgctgcacatgtggcgcacgcagcccatgtcgcccatgccgctgctatggagttgcagcacagaagcaaggaaccaccgccgccagagatcagtgtgtcacgtaagacgcccaaccaatacgaggtggtagacgccagtggtcggcgctcagctggcagtggttccgtttcggtttccgtttcgggcgccaatagccaccattcgccgtatcatccaccggcggcggcctatgcccccagcacctatgccttcccgtacagcgccctgaatgtgcccggtgccgccggtggattgccaccgcaccagccgttgcagctagcccaccaggcggtggcaccacctggtgcctttgccaaggccaaggcagcgcatgccctgagtgaactgggtgcagtcggtggtggggtgtcattggtggtgggcggcggctctggaggaattgcaggcggaccaggtggtgtctcagtcggtgtcggtgtaccgggcggcggcggaccaggaagcggtggctgc

SEQ ID NO: 74 - DNA, 629 bp, Drosophila melanogaster
  misc_feature (1)...(629): n = A,T,C or G
atcaatgctctatgctactatatcttgccttttactataactcgtcgcagctccgacgaacaggaatgtcaggcctgccaatcagtgtcctcggtcatcatgatggtgctccagtactccaacaatccagcgcatcattgccagctcctggagtgcctgatgactcttaagcacaatgtcgtcaaggacatcctctgcgttgtggcatacggaaccgctgtttcccgcacctcggctgccaagctgctcttctactactggccagcctttaacgccaatctgttcgatcgcaaagtcctactctccaaactaaccaatgacctagtgcccttcacctgccaacgggagcactgtccgaactccgggaatgcggaggcagcaaaggtgtgctacgaccacagcattagcatcgcatacgcgcccgattgtccaccgcccctttacctgtgcatcgagtgcgccaacgagattcatcgggagcacggaagcctggagttcggcgacattctgcatcccatgcagcaggtatcgatggtgtgcgaaaacaagaactgtcgctccaacgagaagtccgncttcttcatctgcttttccacggagtgtgccagcttcaatggccaccatccgat

SEQ ID NO: 75 - DNA, 588 bp, Drosophila melanogaster
agagagacaacgacacgacacgacataagtgggggtgggggatagcgaacgagcccatccagcaacaaacttcgcgaacggcggcgacgacgcgcaaagctcgactgaattccaattcgaattcgggcacgctcagaagtaccgttggagtgcagcgacgccggcgatgggtaaacaaaaataggaatggctaaagacgtgcggagcccttgcgctcctccagcccccgtttccgaccctccccccgctgccgctcccgctccaaagacacactcctacaaagagctcaactgtttacacacacacacacacacacaggcacggacacggaagtgtgtatgggtgagacgtaattaaagcttgaaaccgagtttacaacaacaacgagcccgccagtcgccacccaccaccccacgccgcacaccccctgcgaagagccgaagtcgaagcaacagctagaagaagaggcttaagagagagagagagagagagagagagagagagcgggaaagagggaaaattggatacttcgcgcagagagaaacccccaacaacgagcgcagtttataaataaaccttgttcttttc

SEQ ID NO: 76 - DNA, 579 bp, Drosophila melanogaster
tttggctaaccatttctttttatataaaagtaagtaaactaagaactaatcctaggcctgcaggaagtctccgagattgccacatattttgtcgatttccgcacatcccgattgctccagcgctgaaatggcattggcgagggccacggtttctttcagggaatgggccttcaaccatatcctgccgttgactcccacagcgatctcgtagggcagttcccgggtaagagcggcgagaacagggcagttttcccgcagcagcatccttcccagattcaggctgcacttgaagaagaatccatcgatagacatgcaatccacagctacgtgtggatcccgattactctaaccttgtgcgaaggtcaattttccccaaaaaatataggaaacgtaccagggaaaacaacaaaaaagggaaagcgcacccccacactgaaaaccggcgagcacctggaaacgcatacatataaaaggagagtaaatatacaaattggtagcactttcgccgccgtcttttacacattcaagccatgtcttggaccgcttcagttttcttgaggacttacaccactagcatga

SEQ ID NO: 77 - DNA, 656 bp, Drosophila melanogaster
attatgttcagaaccttccgcccggagtcatcgaagtgggtggtctccacatcaagaaccagaccagccctttgcccacgtatatacaagaattcacggagaagttcttcgacggcattgtgtacatcaatatgccctatattgagtatatgaatgaccagggattgaaggctatgtatacgatgattcacggaaatcccaatgttgccttcatttggaatgtggagcaactagagcagttgccggccaagaaaccaaatctgttgacgcttcatgtgaatcaatcactacagcaagacatcttggctatgcagtacgtcaaggggttcctgaatcatggagatagtttcagtcttcaggaggcaattcactatggagtgcccgtcgtcgtgcttccccttaaactagaggaatttaataatgcccaacgtgtaatggaacgcaacttgggtgtgatgcttcaggtcaaggaatttaaccaaagctccctgtcggatgcccttacgcgaatcctggatgaggagcgtttcataagtgctctccaccaggcccagttgaagttccggacccgtccgcaatccgccctggaattggctgtatggcatgcggaacaacttatcgccgaaccacgactatttaaacattttgcacaaact

SEQ ID NO: 78 - DNA, 549 bp, Drosophila melanogaster
  misc_feature (1)...(549): n = A,T,C or G
caacttcgatcggggcatataaaaccagtgcttccaatccgaaggcaaagcataaaagatcagaacatcagtagccgaagattggctgagtagcacggacagcgggcaagtcctttgaaacgttggtagtttgcaaccgggtttgccaacttcctttggagttcagtggtgctcaactatcgacacaactatcctcggctttcgcaaaactcagtaaaccgacacattgacattcgaaaattgggattgaaaactcaagatgccgactacaccacaggatcttgncccttgcccactctcttgctcaaagacctccgaccgatagcagtgaggccaaggagcaggaggccggcgaatcggacaacctgcccaacctgtgcactttgtcgctggacgaactgaaacagctggacagggatcccgagttcttcgaggacttcatcgaggagatgtccgtggtgcagtacctgaacgaggagctcgattcaatgatggaccaggtggagattatatcaagagagaacgagtgcaagggcattcatctggtag

SEQ ID NO: 79 - DNA, 486 bp, Drosophila melanogaster
ccgtcggacagctccgactcggacatctcactgggcacccactcgccggtgccgagcagcctgcagctgcagcatagtccgggcagcacctccaacggcgccaacgaccgcgaggagagcttgagcgtggacgacgacaagccgcgggatctgagcggatcgctgccactgcccctctcgctgcccctgccgctggcctcgcccacccacacgccgccccaactgccgccgggctacgggggcggggcgggcgcaggacccggaggacctctgaccggtccgggctgtctgccacccttcaagctggacgcggtcaccagtctgttcagcgccggctgttacctgcagagcttcagcaacctgaaggagatgtcgcagcagtttcccatccagccgattgtcctgcgtccgcatacgcagctgccgcagtcgctggcactgaacggcgcatccggcggaccgacactgcatcacccggcctacgcg

SEQ ID NO: 80 - DNA, 590 bp, Drosophila melanogaster
aaaggggaaaggcaggcttataagatgagtaaaacgagcttagcgacgcagaccaatggcacggcggaaaatggaaactgttttgatatcctcaaagtgtctaatttgttcgaaaccagccttctggacgaggatgacgtacaattagacgcatacttggcagcctacgaggaaataatgaagttcttccagctcatgggcagtgtcttcagttttgtcagcagcgatgtgcgcagtaaaatagatatcttatacgccctgagagccaaggacgcggaggagcaggaacactttaataccttcagaaccatgctggattacgagaaggaggcccagttgcttactcagaagggttacgtgtctggcagtcgaacgctgctacgtcttcatcgcggtcttgactttgtctacgagtttctcaatcggatccaggcgatacccgacgaccaaaagactgtggacgtgtgcaaggaggcctacgatgacaccctgggcaagcatcactcattcctcatccgcaaaggagcgcgcctggctatgtacgcgatgcccactaggggagatcttctcaagaaagtgtgctccgatgt

(19) United States
(12) Patent Application Publication
(10) Pub. No.: US 2002/0174447 A1
(43) Pub. Date: Nov. 21, 2002

(54) Method for functional mapping of an alzheimer's disease gene network and for identifying therapeutic agents for the treatment of alzheimer's disease

(76) Inventors: Ralph J. Greenspan, Coronado, CA (US); Gerald M. Edelman, La Jolla, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/768,020

(22) Filed: Jan. 23, 2001

(51) Int. Cl. A01K 67/00; G01N 33/00

(52) U.S. Cl. 800/12; 800/3

(57) ABSTRACT

The present invention provides a novel genetic method for mapping a network of functional gene interactions relating to Alzheimer's disease. Further provided by the invention is a screening method for identifying therapeutic agents for treating Alzheimer's disease.

This application is based on, and claims the benefit of, U.S. Provisional Application No. ______ (yet to be assigned), filed Jan. 24, 2000, which was converted from U.S. Ser. No. 09/490,243, and entitled METHOD FOR FUNCTIONAL MAPPING OF AN ALZHEIMER'S DISEASE GENE NETWORK AND FOR IDENTIFYING THERAPEUTIC AGENTS FOR THE TREATMENT OF ALZHEIMER'S DISEASE, and which is incorporated herein by reference.

### BACKGROUND OF THE INVENTION

1. Field of the Invention

The invention relates generally to the fields of genetics and molecular biology and more specifically to a method of using Drosophila to map the network of genetic interactions relating to Alzheimer's Disease.

2. Background Information

Alzheimer's disease, the most common neurodegenerative disease in the world, is a progressive disease that attacks the brain and results in impaired memory, thinking and behavior. Approximately 4 million Americans have Alzheimer's disease and, unless a cure or prevention is found, it is estimated that by the middle of this century 14 million Americans will suffer from the disease. The average lifetime cost per patient is approximately $174,000, with Americans spending at least $100 billion a year on Alzheimer's disease. The only two drugs currently approved by the FDA for treatment of Alzheimer's disease work to temporarily relieve some symptoms but do not extend life expectancy, which is an average of eight years from the onset of symptoms. Thus, there is at present no medical treatment available to cure or stop the progression of Alzheimer's disease.

The human amyloid protein precursor (APP) is centrally implicated in Alzheimer's disease, in part as the source of the amyloid-rich plaques characteristic of this disease. In individuals suffering from Alzheimer's disease, beta-amyloid peptides accumulate and aggregate to form plaques in the brain parenchyma and around blood vessels. In addition, certain APP alleles are known to predispose individuals to Alzheimer's, further implicating the amyloid protein precursor in this disease. However, the normal function of APP is currently unknown; moreover, very little is known about the network of genes that interact with APP or other genes involved in Alzheimer's disease. Understanding the network interactions of genes that directly or indirectly interact with APP is a critical step towards identifying new drug targets, assessing their likelihood of success, and augmenting the efficacy of existing drugs for the treatment of Alzheimer's disease.

Malleable genetic organisms such as Drosophila melanogaster provide convenient experimental systems to study functional gene interactions. That Drosophila can be used to elucidate a human genetic network is supported by the fundamental conservation of cellular mechanisms between humans and Drosophila. Conserved cellular mechanisms include homologous signal transduction pathways, such as the receptor tyrosine kinase activation of Ras and mitogen-activated protein (MAP) kinase (Engstrom et al., Curr. Topics Dev. Biol. 35:229-261 (1997)) and the cAMP activation of protein kinase A (PKA) and cAMP-responsive element-binding protein (CREB) (Dubnau and Tully, Ann. Rev. Neurosci. 21:407-444 (1998)). The fundamental conservation of cellular mechanisms between humans and Drosophila also is supported by mutants such as the fly very long chain fatty acids (VLCFA) acyl CoA synthetase mutant, which shares molecular and phenotypic similarity to the neurodegeneration produced in human adrenoleukodystrophy (Min and Benzer, Science 284:1985-1988 (1999)). Furthermore, the use of Drosophila for elucidating the gene network involved in human Alzheimer's disease is supported by the identification of a Drosophila homolog of human APP. The Drosophila homolog, β amyloid protein precursor-like gene (Appl), is homologous in sequence and function to human APP (Rosen et al., Proc. Natl. Acad. Sci. 86:2478-2482 (1989); Luo et al., Neuron 9: 595-605 (1992)). In view of the above, a lower genetic system such as Drosophila, which carries a gene homologous to a human disease gene, can provide a valuable model system for the study of the functional networks underlying human diseases such as Alzheimer's.

Thus, there is a need for identification of new genes involved in Alzheimer's disease and for a means of mapping the network interactions involved in this disease. The ability to elucidate the genetic network involved in Alzheimer's disease would provide a means of identifying new drug targets and of increasing our understanding of the genetic interactions underlying undesirable side effects. The present invention satisfies this need, relying on the powerful genetics of an experimental genetic system such as Drosophila to provide a method for determining the functional network of genetic interactions underlying Alzheimer's disease. Related advantages are provided as well.

### SUMMARY OF THE INVENTION

The present invention provides a method of mapping a network of functional gene interactions relating to Alzheimer's disease. The method includes the steps of (a) performing matings between (1) a first parent strain carrying a mutation in the Alzheimer's disease gene and (2) a series of parent strains, each containing one of a series of genetic variations, to produce a series of test progeny, where each of the test progeny carry a mutation in the Alzheimer's disease gene and one of the series of genetic variations; and (b) screening the series of test progeny for an altered phenotype relative to at least one sibling control, thereby localizing a gene that is a member of an Alzheimer's disease genetic network to one of the series of genetic variations. In one embodiment, a method of the invention further includes the step of identifying the gene that is a member of an Alzheimer's disease genetic network. In another embodiment, the steps of the method are iteratively repeated in order to identify a network of functional gene interactions relating to Alzheimer's disease.

The methods of the invention can be conveniently practiced by assaying for an altered phenotype such as altered viability, morphology or behavior in test progeny produced by mating two parent strains of, for example, Drosophila melanogaster. In a method of the invention, the Alzheimer's disease gene can map to the X-chromosome or an autosome and can be, for example, amyloid precursor protein-like (Appl) or presenilin (Psn). The mutation can be, for example, an amorph, hypomorph, antimorph, hypermorph or neomorph, and the series of genetic variations can contain, for example, at least twenty or at least one hundred genetic variations. In one embodiment, one or all of the genetic variations map to the X-chromosome. In another embodiment, one or all of the genetic variations map to the autosomes or to one particular autosome.

The present invention also provides a method of identifying a therapeutic agent for treating Alzheimer's disease. The method includes the steps of (a) producing test progeny by performing matings between a first parent strain carrying a mutation in an Alzheimer's disease gene and a second parent strain containing a genetic variation where, in the absence of an agent, the parent strains produce test progeny having an altered phenotype relative to at least one sibling control; (b) administering an agent to the first or second parent strain or the test progeny; and (c) assaying the test progeny for the altered phenotype, where a modification of the altered phenotype producing a phenotype with more similarity to a wild type phenotype than the altered phenotype has to the wild type phenotype indicates that the agent is a therapeutic agent. An Alzheimer's disease gene useful for identifying a therapeutic agent in a method of the invention can be, for example, Appl or Psn. An altered phenotype to be assayed can be, for example, increased or decreased viability in a species such as Drosophila melanogaster.

The invention additionally provides an isolated nucleic acid molecule which is differentially expressed in Appl^{d} versus Appl⁺ Drosophila melanogaster and contains a nucleic acid sequence having substantially the sequence of one of SEQ ID NOS: 1 to 63. Such a differentially expressed nucleic acid molecule can have, for example, the sequence of one of SEQ ID NOS: 1 to 63. Also provided herein is an isolated nucleotide sequence that contains at least 10 contiguous nucleotides of the nucleic acid sequence of one of SEQ ID NOS: 1 to 63.

Further provided herein is an isolated nucleic acid molecule which is differentially expressed in Appl^{d} versus Appl⁺ Drosophila melanogaster and contains a nucleic acid sequence having substantially the sequence of one of SEQ ID NOS: 64 to 80. Such an isolated nucleic acid molecule can have, for example, the sequence of one of SEQ ID NOS: 64 to 80. The invention additionally provides an isolated nucleotide sequence containing at least 10 contiguous nucleotides of the nucleic acid sequence of one of SEQ ID NOS: 64 to 80.

### BRIEF DESCRIPTION OF THE DRAWINGS

FIG. 1 shows the network of genes that interact with Appl, the Drosophila homolog of human APP. In the schematic, “+” denotes increased viability and, denotes decreased viability in comparison to the indicated mutation alone or Appl⁻ alone.

FIG. 2 shows MALDI-TOF analysis of tryptic digests of proteins A1.1, A1.2, A1.3, A1.5, A1.7 and A1.9, which are differentially expressed in Appl^{d} versus Appl⁺ Drosophila.

FIG. 3 shows MALDI-TOF analysis of tryptic digests of proteins A1.12, A1.13, A1.14, A1.15, A1.16 and A1.17, which are differentially expressed in Appl^{d} versus Appl⁺ Drosophila.

FIG. 4 shows MALDI-TOF analysis of tryptic digests of proteins A1.18, A1.21, A1.22, A1.23, A1.24, and A1.26, which are differentially expressed in Appl^{d} versus Appl⁺0 Drosophila.

FIG. 5 shows MALDI-TOF analysis of tryptic digests of proteins A1.27, A1.28, W1.1, A1.2, W1.3 and W1.4, which are differentially expressed in Appl^{d} versus Appl⁺ Drosophila.

FIG. 6 shows MALDI-TOF analysis of tryptic digests of proteins W1.5, W1.6, W1.7, W1.9, W1.10 and W1.11, which are differentially expressed in Appl^{d} versus Appl⁺ Drosophila.

FIG. 7 shows MALDI-TOF analysis of tryptic digests of proteins W1.12, W1.14, W1.15, W1.17, W1.20, W1.21 and W1.22, which are differentially expressed in Appl^{d} versus Appl⁺ Drosophila.

FIG. 8 shows MALDI-TOF analysis of tryptic digests of proteins W1.23 and W1.24, which are differentially expressed in Appl^{d} versus Appl⁺ Drosophila.

### DETAILED DESCRIPTION OF THE INVENTION

The outpouring of new data on human genetics and genome content accentuates the need for a new approach to the problem of understanding gene networks, particularly as these networks relate to diseases such as Alzheimer's disease. Whereas sequence data can provide clues that indicate the physiological role of encoded proteins, malleable experimental systems such as the fruit fly, Drosophila melanogaster, provide a means of systematically analyzing the functional interrelationships of groups of gene products.

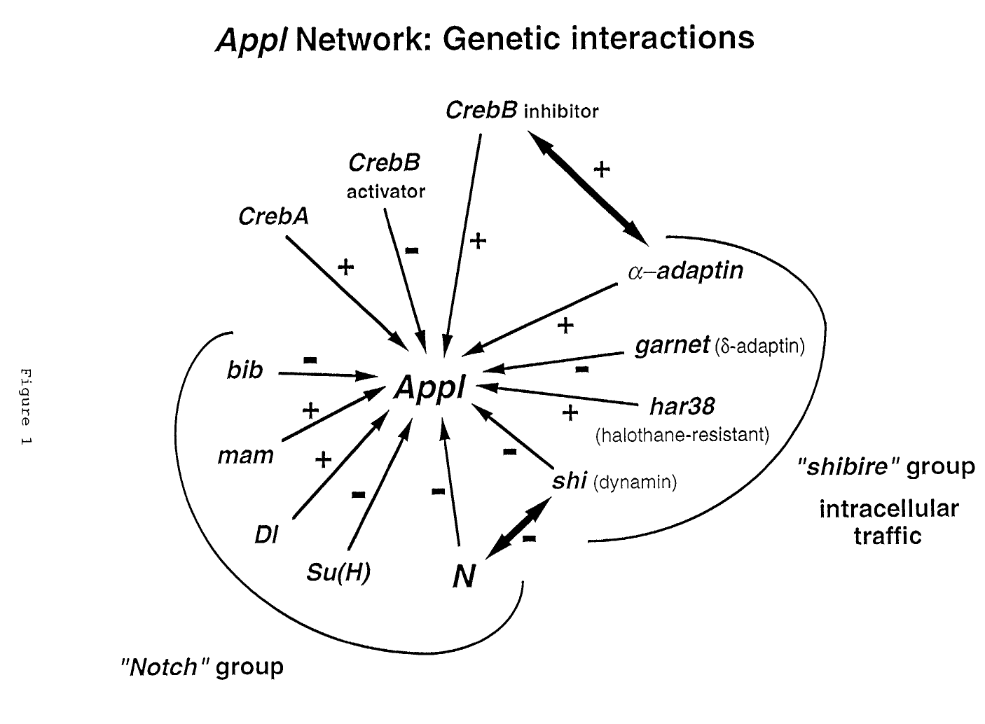

The present invention is directed to a rapid new means of mapping interactions in a gene network. This method, which relies on the powerful genetics of Drosophila and the extensive homology between human and Drosophila genes, is useful for detecting both direct and indirect gene interactions. As disclosed herein, flies bearing a chromosome that lacks the Drosophila homolog of human amyloid precursor protein, Appl (w Appl^{d}), were crossed with a series of flies bearing 34 individual deficiencies of the X chromosome, “Df(1)s.” Subsequently, the number and genotype of adults emerging from each cross were scored, and the viability of flies bearing both the Appl^{d} mutation and the deficiency calculated relative to sibling controls. As shown in Table 1, in 34 combinations of X chromosomal deficiencies with the Appl^{d} mutant, most test progeny had approximately normal viability, five had severely reduced viability (less than 35%), seven had moderately reduced viability, and three had increased viability relative to the controls. Thus, as disclosed herein, the chromosomal segments including 3C2;3E4, 17A1;18A2, 12D2;13A5 and 18E-20 were identified as containing one or more genes that is a member of Alzheimer's disease genetic network. As further disclosed herein, several mutant alleles of genes lying within these chromosomal segments increased or decreased viability in combination with Appl^{d}, thus identifying these genes as members of the genetic network involved in Alzheimer's disease (see Example I). Three prominent gene groupings were identified on the basis of altered viability when combined with Appl^{d}: genes related to the dynamin-encoding shibire; genes relating to Notch, the Drosophila homolog of a human gene implicated in a form of hereditary degenerative dementia; and Creb-related genes, which encode transcription factors implicated in neuronal plasticity and long-term memory formation (see FIG. 1).

TABLE 1
Viability of X Deficiencies with Appl^{d}

| | | | % viability¹ |
|---|---|---|---|
| Df (1) | Breakpoints | N | (1 dose Appl⁺) |
| AD11 | 1B; 2A | 241 | 92.8 |
| A94 | 1E3-4; 2B9-10 | 109 | 94.6 |
| 64c18 | 2E1-2; 3C2 | 144 | 87.0 |
| JC19 | 2F6; 3C5 | 232 | 26.1** |
| N8 | 3C2-3; 3E3-4 | 461 | 32.1** |
| cho2 | 3E; 4A | 236 | 112.6 |
| JC70 | 4C15-16; 5A1-2 | 556 | 103.6 |
| C149 | 5A8-9; 5C5-6 | 312 | 110.8 |
| N73 | 5C2; 5D5-6 | 662 | 104.9 |
| JF5 | 5E6; 5E8 | 1060 | 127.4** |
| 5D | 5D1-2; 5E | 827 | 95.9 |
| Sx1-bt | 6E2; 7A6 | 1454 | 81.5** |
| Ct4b1 | 7B2-4; 7C3-4 | 214 | 18.8** |
| C128 | 7D1; 7D5-6 | 551 | 72.7** |
| KA14 | 7F1-2; 8C61 | 40 | 68.6 |
| 1z-90b24 | 8B5-6; 8D8-9 | 62 | 34.7** |
| 9a4-5 | 8C7-8; 8E1-2 | 145 | 68.6* |
| v-L15 | 9B1-2; 10A1-2 | 542 | 75.9** |
| HA85 | 10C1-2; 11A1-2 | 89 | 71.1 |
| N105 | 10F7; 11D1 | 58 | 45.0** |
| JA26 | 11A1; 11D-E | 136 | 86.3 |
| N12 | 11D1-2; 11F1-2 | 512 | 99.2 |
| KA9 | 12E2-3; 12F5-13A | 196 | 104.1 |
| RK2 | 12D2-E1; 13A2-5 | 241 | 122.4 |
| RK4 | 12F5-6; 13A9-B1 | 408 | 91.5 |
| Sd72b | 13F1; 14B1 | 205 | 70.8* |
| 4b18 | 14B8; 14C1 | 826 | 110.7 |
| N19 | 17A1; 18A2 | 697 | 62.8** |
| JA27 | 18A5.18D | 530 | 91.3 |
| HF396 | 18E1-2; 20 | 47 | 11.9** |
| ma117 | 19A2-3; 19E1 | 106 | 96.2 |
| 2/19B | 19F3-4; 19F6 | 708 | 125.4** |
| JC4 | 20A1; 20EF | 604 | 92.3 |
| A209 | 20A; 20F | 1030 | 87.9 |

w Appl^{d}/Y males were mated to Df (1)/FM7 virgin females and the female progeny counted.
“Df (1)” is the name of the deficiency. “Breakpoints” indicates the chromosomal segment deleted. “N” is the total number of progeny counted.
¹% viability = [(# Df (1)/w Appl^{d})/(#FM7/w Appl^{d})] × 100
*indicates significant departure (P < 0.05) from 100% by X² test
**indicates significant departure (P < 0.01) from 100% by X² test

While APP has been implicated in the pathology of Alzheimer's, its normal biological role has heretofore been unclear. In vitro studies have implicated the protein in neuron-substrate adhesion (Koo et al., Proc. Natl. Acad. Sci. 90:4748-4752 (1993) and Coulson et al., Brain Res. 770:72-80 (1997)); neurite extension (Mattson, J. Neurobiol. 25:439-450 (1994) and Perez et al., J. Neurosci. 17:9407-9414 (1997)); response to copper toxicity or oxidative stress (White et al., J. Neurosci. 19:9170-9179 (1999) and in synaptic plasticity (Roch et al., Proc. Natl. Acad. Sci., USA 91:7450-7454 (1994); Ishida et al., NeuroReport 8:2133-2137 (1997)). Studies of the Appl^{d} mutant in Drosophila have supported its involvement in synaptic plasticity and differentiation (Torroja et al., Curr. Biol. 9:489-492 (1999)). Furthermore, overexpression of Appl⁺ in Drosophila in conjunction with expression of human tau, produces developmental defects including a disruption of axonal transport (Torroja et al., J. Neurosci. 19:7793-7803 (1999)).

The functional gene interactions disclosed herein indicate that the APPL protein in flies, and similarly the human APP protein, is involved in vesicle endo- and exo-cytosis. Such a role is supported, for example, by the disclosed interactions of Appl with shibire, α-adaptin and garnet (δ-adaptin). Such a role also is supported by interactions with halothane-resistant mutants, given that anesthesia-resistant mutants in C. elegans affect the vesicle fusion machinery (van Swinderen et al., Proc. Natl. Acad. Sci., USA 96:2479-2484 (1999)). In addition, a role for APP in vesicle endo- and exocytosis is supported by several genes that are differentially expressed in Appl^{d} versus wild type flies, for example, exo84, Frequenin, 14-3-3ζ, PPA2a2, myosin-IB and actin57B (see below). Furthermore, the interactions of Appl with Notch and its gene group can be related to the requirement for precise and reciprocal regulation of the quantities of Notch and Delta proteins on the membranes of neighboring cells during development (Heitzler and Simpson, Cell 64:1083-1092 (1991)), given that endocytosis can be the mechanism of their down-regulation.

Where normal APP protein is involved in the actual process of vesicle cycling, APP mutants can render the actual machinery abnormal, not merely the final disposition and clearing of this particular protein and its derivatives. In view of the viability of null mutants for Appl in flies and APP knockouts in mice, the APP mutants do not result in a major defect in vesicle cycling. Thus, the disclosed methods for mapping functional gene interactions can elucidate the normal biology of critical genes as well as their role in pathogenesis.

The present invention provides a method of mapping a network of functional gene interactions relating to Alzheimer's disease. The method includes the steps of (a) performing matings between (1) a first parent strain carrying a mutation in the Alzheimer's disease gene and (2) a series of parent strains, each containing one of a series of genetic variations, to produce a series of test progeny, where each of the test progeny carry a mutation in the Alzheimer's disease gene and one of the series of genetic variations; and (b) screening the series of test progeny for an altered phenotype relative to at least one sibling control, thereby localizing a gene that is a member of an Alzheimer's disease genetic network to one of the series of genetic variations. In one embodiement, a method of the invention can further include the step of identifying the gene that is a member of an Alzheimer's disease genetic network. In another embodiment, the steps of the invention are iteratively repeated in order to identify a network of functional gene interactions relating to Alzheimer's disease.

The methods of the invention can be conveniently practiced by assaying for an altered phenotype such as altered viability, morphology or behavior in test progeny produced by mating parent strains of, for example, Drosophilidae such as Drosophila melanogaster. In a method of the invention, the Alzheimer's disease gene can map to the X-chromosome or an autosome and can be, for example, amyloid precursor protein-like (Appl) or presenilin (Psn). The mutation can be, for example, an amorph, hypomorph, antimorph, hypermorph or neomorph, and the series of genetic variations can contain, for example, at least twenty or at least one hundred genetic variations. In one embodiment, one or all of the genetic variations map to the X-chromosome. In another embodiment, one or all of the genetic variations map to the autosomes or to one particular autosome.

As used herein, the term “Alzheimer's disease gene” means a homolog of a human gene that has genetic variants associated with an increased risk of Alzheimer's disease or that encodes a gene product associated with Alzheimer's disease. While Appl and Presenilin (Psn) are provided herein as Alzheimer's disease genes useful in the invention, one skilled in the art also can practice the invention with one of a variety of other Alzheimer's disease genes. Additional exemplary Alzheimer's disease genes include the genes identified herein as interacting (directly or indirectly) with Appl: Notch (N), Suppressor of Hairless (Su (H)), Delta (Dl), mastermind (mam), big brain (bib), halothane resistant (har38), cAMP-responsive element-binding protein A (CrebA), cAMP-responsive element-binding protein B (CrebB, activator), cAMP-responsive element-binding protein B (CrebB, inhibitor), α-adaptin, garnet (δ-adaptin), and shibire (shi) (dynamin). In addition, an Alzheimer's disease gene can be a gene that is differentially expressed at the mRNA or protein level in Appl^{d} flies as compared to Appl⁺ flies as described further below (see Tables 4-6). One skilled in the art understands that these or other Alzheimer's disease genes can be used to practice the methods of the invention. Generally, an Alzheimer's disease gene will encode a polypeptide having at least about 25%, 30%, 40%, 50%, 75% or greater amino acid identity with its human homolog and will share one or more functional characteristics with its human homolog. Methods for cloning homologs of human genes using routine methods such as PCR or library screening are well known in the art as described, for example, in Ausubel et al., Current Protocols in Molecular Biology, John Wiley and Sons, Baltimore, Md. (1998).

The term “mutation,” as used herein in reference to an Alzheimer's disease gene, means a stably inherited change in the primary nucleic acid sequence of the Alzheimer's disease gene. Preferably, the mutation is restricted to the Alzheimer's disease gene and does not affect additional genes. As used herein, the term mutation encompasses genetic lesions that result in a complete or partial loss of function, a function that is antagonistic to the activity of the wild type protein, increased function or gain of a novel function. The designation Appl^{d} represents a chromosome lacking the Appl gene and is an exemplary mutation in an Alzheimer's disease gene. The designation Appl⁻ is used herein to refer to any null or hypomorphic mutation of Appl (see below).

The term “amorph,” as used herein, means a mutation that completely eliminates the function of a gene product and is synonymous with “null mutation.” An amorph or null mutation can be produced, for example, by partial or complete deletion of a gene, by a molecular lesion that blocks transcription or translation, or by a nonsense or missense mutation or other lesion within the coding sequence.

The term “hypomorph,” as used herein, means a mutation that results in a partial loss of function of an Alzheimer's disease gene product. A hypomorph can be, for example, a mutation that reduces the expression, stability or activity of the encoded gene product.

The term “antimorph,” as used herein, means a mutation that is antagonistic to the activity of the encoded wild type gene product and is synonymous with “dominant negative mutation.”

As used herein, the term “hypermorph” means a mutation that results in increased function of the encoded Alzheimer's disease gene product. A hypermorph can be, for example, a mutation that enhances the expression, stability or activity of the encoded gene product.

The term “neomorph,” as used herein, means a mutation that results in a novel function of the encoded gene product and is synonymous with “gain-of-function mutation.” Such a novel function can occur as a consequence, for example, of ectopic expression of the encoded gene product.

While the methods of the invention are exemplified herein using the genetic system Drosophila, any genetic system suitable for transmission genetics and convenient analysis of test and sibling control progeny is useful for practicing the methods of the invention. Examples of genetic systems suitable for practicing the methods of the invention include, for example, mice (Mus musculus), zebrafish (Danio rerio), nematodes (Caenorhabditis elegans), and yeast (Saccharomyces cerevisiae and Schizosaccharomyces pombe). Homologs of human disease genes have been identified in each of these species. For example, the murine frizzled gene is the homolog of human FZD9 deleted in Williams-Beuren syndrome (Wang et al., Genomics 57:235-248 (1999). In zebrafish, homologs of human genes implicated in, for example, Huntington's disease or congenital sideroblastic anemia have been isolated and characterized (Karlovich et al., Gene 217: 117-125 (1998); and Brownlie et al., Nat. Genet. 20(3):244-50 (1998)). In C. elegans, Alzheimer's disease genes include vab-3, a homolog of PAX-6, which is associated with aniridia in humans; and sma-4, a homolog of the pancreatic carcinoma gene DPC4 (Ahringer, Curr. Opin. Genet. Dev. 7:410-415 (1997) and the Presenilin homologs spe-4 and sel-12 (Hutton and Hardy, Human Molecular Genetics 10:1639-1646 (1997); and Levitan and Greenwald, Nature 377:351-354 (1995))). In yeast (S. cerevisiae and S. pombe) and mice, homologs of the human RAD30 gene implicated in xeroderma pigmentosum have been identified (McDonald et al., Genomics 60:20-30 (1999). Methods of performing matings and analyzing progeny in mice, zebrafish, nematodes and yeast are well known in the art (see Jackson, for example, Mouse Genetics and Transgenics: A Practical Approach, Oxford University Press, Oxford, U.K. (2000); Detrich et al., The Zebrafish: Genetics and Genomics, Academic Press, San Diego (1998); Hope, C. Elegans: A Practical Approach, Oxford University Press, Oxford, U.K. (1999); and Adams et al., Methods in Yeast Genetics, 1997: A Cold Spring Harbor Laboratory Course Manual, Cold Spring Harbor Laboratory Press, Cold Spring Harbor, N.Y. (1997)).

The methods of the invention have been exemplified herein using Appl in order to map a network of functional gene interactions relating to Alzheimer's disease. However, the methods of the invention are equally applicable to mapping a network of functional interactions relating to another human disease. As set forth in Table 2, Drosophila homologs of a variety of human disease genes are available, including genes involved in sclerosis such as tuberous sclerosis, amyotrophic lateral sclerosis; diseases of the eye such as aniridia, cataract, glaucoma, nystagmus, atypical colobomata, slitlike iris, stromal defects, optic nerve hypoplasia; and numerous other diseases such as Huntington's disease, retinitis pigmentosum, Waardenburg syndrome (Type 1), basal cell nevus syndrome, adenomatous polyposis of the colon, holoprosencephaly (Type 3), myotonic dystrophy, atrial septal defect with atrioventicular conduction defects, hyperprolinemia (Type 1), branchiooterenal dysplasia, renal-coloboma syndrome, selective tooth agenesis, X-linked hydrocephalus, Masa syndrome, spastic paraplegia 1, oroticaceduria 1, Saethre-Chotzen syndrome, Greig cephalopolysyndactyly, Pallister-Hall syndrome, postaxial polydactyly type A, parathyroid adenomatosis 1, SCID, cerebral autosomal dominant arteriopathy, multiple endocrine neoplasia (Type IIA), Hirschsprung disease, mesothelomia, WAGR syndrome, juvenile polyposis syndrome, neurofibromatosis, neurofibromatosis Type II, Cowden disease, Lhermitte-Duclos disease, Bannayan-Zonana syndrome, xeroderma pigmentosum A, xeroderma pigmentosum B, xeroderma pigmentosum D, Angelman syndrome and Vohwinkel syndrome. Table 2 further describes Drosophila homologs of genes involved in cancers such as alveolar rhabdomyosarcoma, sporadic basal cell carcinoma, colorectal cancer, hepatoblastoma, chronic myelogenous leukemia, T-cell leukemia, gastric adenocarcinoma, ovarian and pancreatic carcinoma, malignant melanoma, B-cell lymphoma, retinoblastoma, pre-B cell acute lymphoblastic leukemia, acute lymphoblastic leukemia, non-Hodgkin lymphoma, myeloid leukemia, Burkitt's lymphoma, T-cell lymphoblastic leukemia, bladder carcinoma, renal pelvic carcinoma, mammary carcinosarcoma, ovarian cancer, medullary thyroid carcinoma, neuroblastoma, glioblastoma, esophageal carcinoma, Wilm's tumor, lung cancer and sporadic prostate cancer. One skilled in the art understands that additional homologs of human disease genes are known in the art for Drosophila as well as for other species such as mice, zebrafish, nematodes and yeast, or can be identified by routine methods.

TABLE 2

| Fly Gene | Human Gene | Disease/Reference |
|---|---|---|
| huntingtin | HUNTINGTIN | Huntington's disease |
| (AF 177386) | (NM_002111) | (Cell 72:971-983 (1993)) |
| Appl (J04516) | AMYLOID PROTEIN | Alzheimer's disease |
| | PRECURSOR | (Goate et al. Nature |
| | (NM_000484) | 349:704-706 (1991)) |
| Psn (U77934) | PRESENILIN-1 | Alzheimer's disease, familial |
| | (NM_000021) | type 3 |
| | | (Sherrington et al., Nature |
| | | 375:754-760 (1995)) |
| Sod (Z19591) | SUPEROXIDE | Amyotrophic lateral sclerosis |
| | DISMUTASE | (Rosen et al., Nature |
| | (NM_000454) | 362:59-62 (1993)) |
| Eaat1 | AF001784 | Amyotrophic lateral sclerosis |
| | (U03505) | |
| Eaat2 | AF166000 | Amyotrophic lateral sclerosis |
| | (U03505) | |
| gigas | TSC2 (X75621) | Tuberous sclerosis |
| (AF172995) | | (Kumar et al., Hum. Molec. |
| | | Genet. 4:1471-1472 (1995)) |
| NinaE (K02315) | RHODOPSIN | Retinitis pigmentosum |
| | (NM_000539) | (Dryja et al., New Eng. J. |
| | | Med. 323:1302-1307 (1990)) |
| forkhead | FKHR (AF032885) | Rhabdomyosarcoma, alveolar |
| (J03177) | | (Whang-Peng et al., Genes |
| | | Chromosomes Cancer |
| | | 5:299-310 (1992)) |
| paired (M14548) | PAX3 (U12263) | Waardenburg syndrome, |
| | | type 1 |
| | | (Milunsky et al., Am. J. Hum. |
| | | Genet. 51 (suppl.):A222 |
| | | (1992)) |
| patched | PTCH2 | Basal cell nevus syndrome |
| (X17558) | (AF087651) | (Wicking et al., Am. J. Hum. |
| | | Genet. 60:21-26 (1997)) |
| dAPC1 | APC | Adenomatous polyposis of the |
| (U77947) | (NM_000038) | colon |
| dAPC2 | | (Groden et al., Cell |
| (AF113913) | | 66:589-600 (1991)) |
| hedgehog | SONIC HEDGEHOG | Holoprosencephaly, type 3 |
| (L02793) | (NM_000193) | (Roessler et al., Nature Genet. |
| | | 14:357-360 (1996)) |
| Sine oculis | SIX5 (X84813) | Myotonic dystrophy, |
| (L31626) | | ophthalmic aspects |
| | | (Winchester et al., |
| | | Hum. Molec. Genet. |
| | | 8:481-492 (1999)) |
| tinman | CSX (NM_004387) | Atrial septal defect with |
| (AF004336) | | atrioventricular conduction |
| | | defects |
| | | (Schott et al., Science |
| | | 281:108-111 (1998)) |
| sluggish-A | PRODH | Hyperprolinemia, type 1 |
| (L07330) | (NM_005974) | (Campbell et al., Hum. |
| | | Genet. 101:69-74 (1997)) |
| eyeless | PAX6 | Aniridia, cataract, glaucoma, |
| (X79493) | (NM_000280) | nystagmus, atypical |
| | | colobomata, slitlike iris, |
| | | stromal defects, optic nerve |
| | | hypoplasia (Jordan et al., |
| | | Nature Genet. |
| | | 1:328-332 (1992)) |
| clift/eyes | EYA2 | Branchiootorenal dysplasia |
| absent (L08502) | (NM_005244) | (Abdelhak et al., Nature |
| | | Genet. 15:157-164 (1997)) |
| Pox-m (X58917) | PAX2 (AH006910) | Renal-coloboma syndrome |
| | | (Sanyanusin et al., Nature |
| | | Genet. 9:358-364 (1995)) |
| muscle segment | MSX1 (M76731) | Tooth agenesis, selective |
| homeobox | | (Vastardis et al., Nature |
| (X85331) | | Genet. 13:417-421 (1996)) |
| Fasciclin-2 | L1-CAM (Z29373) | Hydrocephalus, X-linked, |
| (M77165) | | Masa syndrome, |
| | | Spastic paraplegia 1 |
| | | (Jouet et al., Nature Genet. |
| | | 4:331 (1993)) |
| rudimentary- | OPRT and OMP | Oroticaciduria 1 |
| like (L00968) | (NM_000373) | (Suchi et al., Am. J. Hum. |
| | | Genet. 60:525-539 (1997)) |
| smoothened | SMOH | Basal cell carcinoma, sporadic |
| (U87613) | (NM_005631) | (Xie et al., Nature 391:90-92 |
| | | (1998)) |
| twist (X12506) | TWIST (Y10871) | Saethre-Chotzen syndrome |
| | | (Krebs et al., Hum. Mol. |
| | | Genet. 6:1079-1086 (1997)) |
| armadillo | CATENIN-β-1 | Colorectal cancer, |
| (X54468) | (NM_001904) | hepatoblastoma, |
| | | pilomatricoma |
| | | (Morin et al., Science |
| | | 275:1787-1790 (1997)) |
| Ab1 (M19690) | ABL1 (Abelson) | Chronic myelogenous |
| | (NM_005157) | leukemia (Chissoe et al., |
| | | Genomics 27:67-82 (1995)) |
| Akt1 (X83510) | AKT2 (M95936) | T-Cell leukemia, gastric |
| | | adenocarcinoma ovarian and |
| | | pancreatic carcinoma |
| | | (Cheng et al., Proc. Natl. |
| | | Acad. Sci. 89:9267-9271 |
| | | (1992)) |
| aurora (X83465) | AIM1 (U83115) | Malignant melanoma |
| | | (Trent et al., Science |
| | | 247:568-571 (1990)) |
| cubitus | GLI3 | Greig cephalopolysyndactyly, |
| interruptus | (NM_000168) | Pallister-Hall syndrome, and |
| (X54360) | | postaxial polydactyly type A |
| | | (Kang et al., Nature Genet. |
| | | 15:266-268 (1997)) |
| cyclin D | CCND1 | Parathyroid adenomatosis 1, |
| (U41808) | (NM-001758) | breast and sqaumous cell |
| | | cancer (Rosenberg et al., |
| | | Oncogene 6:449-453 (1991)) |
| dorsal (M23702) | NFKB1 (M58603) | B-cell lymphoma, inflam- |
| | | mation (Barnes and Karin, |
| | | New Eng. J. Med. |
| | | 336:1066-1071 (1997)) |
| E2F (U10184) | E2F | Retinoblastoma |
| | transcription | (Pan et al., Molec. Cell |
| | factor 1 | 2:283-292 (1998)) |
| | (NM_006286) | |
| extradenticle | PBX1 (M31522) | Pre-B cell acute lymphoblastic |
| (U33747) | | leukemia (Kamps et al., Cell |
| | | 60:547-555 (1990)) |
| hopscotch | JAK3 | SCID, autosomal recessive, |
| (L26975) | (NM_000215) | T-negative/B-positive type |
| | | (Macchi et al., Nature |
| | | 377:65-68 (1995)) |
| Myb (M11281) | MYB (NM_005375) | acute lymphoblastic leukemia, |
| | | non-Hodgkin lymphoma, |
| | | myeloid leukemia, malignant |
| | | melanoma, metastases |
| | | (Linnenbach et al., Proc. Natl. |
| | | Acad. Sci. 85:74-78 (1988)) |
| Myc (K03060) | MYC (X00364) | Burkitt's lymphoma |
| | | (Bhatia et al., Nature Genet. |
| | | 5:56-61 (1993)) |
| Notch | NOTCH1 (M73980) | T-cell lymphoblastic leukemia |
| CM16150/ | | (Ellisen et al., Cell |
| M11664) | | 66:649-661 (1991)) |
| | NOTCH3 (U97669) | Cerebral autosomal dominant |
| | | arteriopathy with subcortical |
| | | infarcts and |
| | | leukoencephalopathy |
| | | (Joutel et al., Nature |
| | | 383:707-710 (1996)) |
| Ras64B (K01961) | HRAS (J00277) | Bladder, lung, renal pelvic |
| | | carcinoma, mammary |
| | | carcinosarcoma, melanoma, |
| | | ovarian and colorectal cancer |
| | | (Taparowsky et al., Nature |
| | | 300:762-765 (1982); Nakano |
| | | et al., Proc. Natl. Acad. Sci. |
| | | 81:71-75 (1984)) |
| Ras85D (K01960) | KRAS (K01912) | Bladder, lung, renal pelvic |
| | | carcinoma, mammary |
| | | carcinosarcoma, melanoma, |
| | | ovarian and colorectal cancer |
| | | (Taparowsky et al., Nature |
| | | 300:762-765 (1982); Nakano |
| | | et al., Proc. Natl. Acad. Sci. |
| | | 81:71-75 (1984)) |
| Ret (D16401) | RET (X12949) | Multiple endocrine neoplasia, |
| | | Type Iia, Hirschsprung di- |
| | | sease, medullary thyroid |
| | | carcinoma |
| | | (Mulligan et al., Nature |
| | | 363:458-460 (1993)) |
| Src42A (D42125) | SRC (NM_005417) | Colon cancer |
| Src64B (K01043) | | (Irby et al., Nature Genet. |
| | | 21:187-190 (1999)) |
| trithorax | ALL1 | Myeloid/lymphoid leukemia |
| (M31617) | (NM_005935) | (Sorensen et al., J. Clin. |
| | | Invest. 93:429-437 (1994)) |
| Egfr (K03054) | ERBB2 | Neuroblastoma, glioblastoma, |
| | (NM_004448) | mammary carcinoma |
| | | (Di Fiore et al., Science |
| | | 237:178-182 (1987)) |
| frazzled | DCC (NM_005215) | Colorectal, esophageal |
| (U71001) | | carcinoma |
| | | (Cho et al., Genomics |
| | | 19:525-531 (1994)) |
| klumpfuss | WT1 (X51630) | Wilms tumor, mesothelioma, |
| (Y11066) | | WAGR syndrome |
| | | (Pelletier et al., Cell |
| | | 67:437-447 (1991)) |
| Medea | MADH4 | Pancreatic carcinoma, juvenile |
| (AF019753) | (NM_005359) | polyposis syndrome |
| | | (Schutte et al., Cancer Res. |
| | | 56:2527-2530 (1996)) |
| Nf1 (L26500) | NF1 (NM_000267) | Neurofibromatosis |
| | | (Upadhyaya et al., Hum. |
| | | Mutat. 4:83-101 (1994)) |
| Merlin (U49724) | NF2 (NM_000268) | Neurofibromatiosis, Type II |
| | | (Rouleau et al., Nature |
| | | 363:515-521 (1993)) |
| PP2A-29B | PPP2R1B | Lung cancer |
| (M86442) | (AF083439) | (Wang et al., Science |
| | | 282:284-287 (1998)) |
| Pten (AF161257) | PTEN | Cowden disease, |
| | (NM_000314) | Lhermitte-Duclos disease, |
| | | Bannayan-Zonana syndrome, |
| | | endometrial carcinoma, |
| | | juvenile polyposis syndrome, |
| | | sporadic prostate cancer |
| | | (Liaw et al., Nature Genet. |
| | | 16:64-67 (1997)) |
| Rbf (X96975) | RB1 (NM_000321) | Retinoblastoma, soft tissue |
| | | carcinomas |
| | | (Harbour et al., Science |
| | | 241:353-357 (1998)) |
| spellchecker | MSH2 | Colon cancer, familial |
| (U17893) | (NM_000251) | nonpolyposis, Type 1 |
| | | (Leach et al., Cell |
| | | 75:1215-1225 (1993)) |
| mus210 (Z28622) | XPA (NM_000380) | Xeroderma pigmentosum A |
| | | (Tanaka et al., Nature |
| | | 348:73-76 (1990)) |
| haywire | ERCC3 | Xeroderma pigmentosum B |
| (L02965) | (NM 000122) | (Weeda et al., |
| | | Cell 62:777-791 (1990)) |
| Xpd (AF132140) | ERCC2 (X52221) | Xeroderma pigmentosum D |
| | | (Frederick, Hum. Molec. |
| | | Genet. 3:1783-1788 (1994)) |
| hyperplastic | UBE3A | Angelman syndrome |
| discs (L14644) | (NM_000462) | (Kishino et al., Nature Genet. |
| | | 15:70-73 (1997)) |
| 1 (3) malignant | LOR (NM_000427) | Vohwinkel syndrome |
| blood | | (Maestrini et al., Nature |
| neoplasm-1 | | Genet. 13:70-77 (1996)) |
| (Z47722) | | |

As used herein, the term “genetic variation” means a stably inherited change in the nucleic acid sequence of genomic DNA. A genetic variation can be a naturally occurring or man-made variation such as a chromosomal deficiency, inversion, duplication or translocation, or a substitution, insertion or deletion of one or more nucleotides. Thus, a genetic variation can be, for example, a substitution, insertion or deletion of 1 to 1000 nucleotides, 1 to 100 nucleotides, 1 to 50 nucleotides or 1 to 10 nucleotides. One skilled in the art understands that a genetic variation also can be a molecular variation such as abnormal methylation or other modification that does not produce a difference in the primary nucleic acid sequence of genomic DNA, provided that such a molecular variation is stably inherited. One skilled in the art understands that an individual heterozygous or hemizygous for a genetic variation may or may not exhibit an altered phenotype relative to its wild type siblings; however, a genetic variation useful in the methods of the invention generally affects the function or expression of an encoded gene product.

A genetic variation can be readily obtained from a variety of public sources or can be routinely prepared using, for example, standard mutagenesis procedures. For example, a series of parent Drosophila strains, each containing one of a series of genetic variations, can be obtained from The Bloomington Drosophila Stock Center at Indiana University (Bloomington, Ind.), a public repository containing about 7000 fly stocks including a variety of deficiency stocks and stocks carrying mutant alleles of particular genes. A complete list of stocks is available on the Internet at http://www.flystocks.bio.indiana.edu.

Mutagenesis, such as radiation, chemical or insertional mutagenesis, also can be used to routinely prepare a series of genetic variations in Drosophila. One skilled in the art understands that the appropriate mutagen will depend, in part, on the desired genetic variation. For example, a chemical mutagen such as ethylmethane sulfonate (EMS) is most suitable for obtaining a point mutation or a small, intragenic deletion, while radiation with X-rays or gamma-rays is most suitable for producing chromosomal rearrangements. Insertional mutagenesis with a transposable element such as a P-element also is useful for producing a series of genetic variations and facilitates rapid molecular analysis of the variations. A detailed account of various Drosophila mutagens, their properties and uses is available, for example, in Ashburner, Drosophila: A Laboratory Handbook, Cold Spring Harbor Press, Cold Spring Harbor, N.Y. (1989).

EMS, an alkylating agent, is particularly useful for chemical mutagenesis in Drosophila. The dose of EMS required to induce a mutation depends on whether the goal is to induce a new mutation or a new allele of an existing mutation. Generally, 3 to 5 day old males are fed a solution of about 2.5 mM to 7.5 mM EMS in 1% sucrose overnight and mated to females for 4 to 5 days, followed by recovery and further test-crossing of the Fl progeny in order to reveal the presence of new mutations (Ashburner, supra, 1989). Appropriate test crosses of the Fl progeny aimed at identification of a new nutation or allele, and recovery of the mutation bearing chromosome are routine in the art as described, for example, in Greenspan, Fly Pushinq: The Theory and Practice of Drosophila Genetics, Cold Spring Harbor Laboratory Press, Plainview, N.Y. (1997).

Radiation also can be used to prepare one or more genetic variations, although generally the frequency of introducing a genetic variation by radiation mutagenesis is considerably lower than for chemical mutagenesis. X-ray machines and cobalt or cesium sources for gamma rays are particularly useful sources of radiation, which, in Drosophila, are typically used to irradiate mature males. Methods of using radiation to produce genetic variations in Drosophila are well known in the art as described, for example, in Kelley et al., Genetics 109:365-377 (1985); Sequeira et al., Genetics 123:511-524 (1989); and Sliter et al., Genetics 123:327-336 (1989);

In addition to chemical and radiation mutagenesis, transposable genetic elements allow for insertional mutagenesis in Drosophila. P-elements, especially “enhancer-trap” P-elements, which express β-galactosidase in a tissue-specific manner depending on the site of insertion, are particularly useful for producing one or more genetic variations in Drosophila (O'Kane et al., Proc. Natl. Acad. Sci. 84:9123-9127 (1987); Bellen et al., Genes Dev. 3:1288-1300 (1989); and Bier et al., Genes Dev. 3:1273-1287 (1989)). In contrast to classical chemical or radiation mutagenesis, insertional mutagenesis with enhancer-trap P-elements provides a means for identifying new genes based on expression pattern rather than mutant phenotype. New insertion lines can be routinely generated and screened based on the position-dependent tissue expression of the lacZ reporter gene. An insertion line showing an expression pattern of interest can be further analyzed to ascertain whether the insertion has disrupted a gene of interest. Methods for insertional mutagenesis utilizing enhancer trap P-elements, including mating schemes appropriate for the identification of newly induced genetic variations, are well known in the art as described, for example, in Bier et al., supra, 1989, and Greenspan, supra, 1997.

Chromosomal deficiencies are genetic variations that can be particularly useful in a method of the invention. As used herein, the term “chromosomal deficiency” is synonymous with “deficiency” or “deletion” and means a rearrangement in which a contiguous portion of a chromosome is excised and the regions flanking the excised portion are joined together, thus excluding the excised portion of the chromosome. A chromosomal deficiency can be a very short excision of only a few nucleotides, or can be large enough to excise, for example, several hundred genes or about 15% of one arm of a Drosophila chromosome.

A series of chromosomal deficiencies can be useful for genetically scanning a significant portion of the genome to map functional gene interactions involved in Alzheimer's disease. As disclosed herein, males carrying Appl^{d} were crossed with a series of females heterozygous for 34 individual deficiencies of the X chromosome (Df(1)s). This series of deficiencies together covers roughly 70% of the X chromosome and nearly 15% of the entire Drosophila genome, thus serving as a representative example of the Drosophila genome. As disclosed herein, several deficiencies, including Df(1)N8, Df(1)N19, Df(1)JC19, Df(1)JF5, Df(1)Sxe-bt, Df(1)ct461, Df(1)c128, Df(1)LZ-90624, Df(1)9a4-5, Df(1)V-L15, Df(1)N105, Df(1)sd72b, Df(1)HF396 and Df(1)2/19B, were identified as containing one or more genes that are members of an Alzheimer's disease gene network based on an altered phenotype of increased or decreased viability when combined with Appl^{d}. One skilled in the art understands that deficiencies which produce an altered phenotype can be further subdivided, if desired, and matings with Appl^{d} flies repeated in order to identify the genes within the chromosomal deficiency that are members of the Alzheimer's disease gene network.

The methods of the invention rely on screening a series of test progeny for an altered phenotype. As used herein, the term “phenotype” refers to the physical appearance or observable properties of an individual that are produced, in part, by the genotype of the individual. A variety of behavioral, morphological and other physical phenotypes are useful in the methods of the invention including Drosophila phenotypes such as eye color, wing shape, bristle appearance, size, phototaxis and viability. Additional phenotypes useful for practicing the invention include the size, viability, eye color, coat color, or exploratory behavior of mice; the size, viability, skin color, or optomotor response of zebrafish; the size, viability, phototaxis or chemotaxis of nematodes; and the colony color, colony size or growth requirements of yeast.

Viability represents a phenotype that is particularly useful for establishing a functional interaction between genes: as disclosed in Example I, flies carrying a combination of Appl^{d} and the chromosomal deficiency Df(1)N8, Df(1)JC19, 9Df(1)ct4b1, Df(1)1z-90b24 or Df(1)HF396 had significantly decreased viability as compared to sibling controls, while flies carrying Appl^{d} and the chromosomal deficiency Df(1)JF5, Df(1)2/19B or Df(1)RK2 had significantly increased viability as compared to sibling controls. As further disclosed in Example III, Appl^{d} Drosophila have a defect in fast phototaxis; such a behavioral phenotype also can be useful in the methods of the invention for establishing a functional interaction as is disclosed herein for Appl and Notch, Delta, α-adaptin, dCrebA and dCrebB. Two mutants (Notch and Delta) showed significant phototaxis interactions with Appl^{d}/+ flies, and three mutants (α-adaptin, dCrebA and dCrebB) showed significant phototaxis interactions with Appl^{d} flies.

The term “altered phenotype,” as used herein in reference to the phenotype of test progeny as compared to a sibling control, means a significant change in the physical appearance or observable properties of the test progeny as compared to a sibling control. Thus, the term altered phenotype is used broadly to encompass both a phenotype that is dramatically changed as compared to the phenotype of a sibling control as well as a phenotype that is slightly but significantly changed as compared to a sibling control.

It is recognized that there can be natural variation in the phenotypes of test progeny. However, an altered phenotype readily can be identified by sampling a population of test progeny and determining that the normal distribution of phenotypes is changed, on average, as compared to the normal distribution of phenotypes in a population of sibling controls. Where a phenotype can be quantified, the alteration will be statistically significant and generally will be an increase or decrease of at least about 5%, 10%, 20%, 30%, 50% or 100% as compared to sibling controls. For example, as disclosed herein in Example I, viability scores less than 80% or more than 110% of sibling controls carrying one copy of Appl^{d} were statistically significant and, thus, are examples of an “altered phenotype.”

As used herein, the term “test progeny” refers to progeny carrying both a mutation in an Alzheimer's disease gene and a genetic variation. Test progeny, which are produced by mating a parent strain carrying a mutation in an Alzheimer's disease gene with a parent strain carrying a genetic variation, may or may not have an altered phenotype.

Test progeny can be doubly heterozygous for a mutation in an Alzheimer's disease gene and for a genetic variation. The term “doubly heterozygous,” as used herein in reference to test progeny, means diploid test progeny with both a single allele of the mutation in an Alzheimer's disease gene and a single allele of a genetic variation.

As used herein, the term “sibling control” means control progeny that are genetically similar to the test progeny and carry either the mutation in an Alzheimer's disease gene or the genetic variation, but not both. The term sibling control encompasses actual siblings produced in the mating giving rise to the test progeny as well as control progeny produced in a parallel mating.

Control siblings can be conveniently identified in Drosophila using balancer chromosomes. As used herein, the term “balancer chromosome” means a multiply inverted Drosophila chromosome usually carrying a dominant marker mutation. One skilled in the art understands that a useful balancer chromosome carries multiple chromosomal inversions and suppresses recombination along the full length of the chromosome. A balancer chromosome also can carry a dominant marker mutation resulting in a phenotype such as a particular eye color or wing phenotype that can be readily identified in flies carrying the balancer. In addition, a balancer chromosome may contain one or more recessive marker mutations for easy identification of progeny carrying two copies of the balancer during segregation analysis. Balancer chromosomes are available for the X, second, and third Drosophila chromosomes: for example, FM7a, FM7b and FM7c are convenient X chromosome balancers; SM6 and In (2LR) O, Cy dp^{1v1} pr cn² are convenient balancers for the second chromosome; and TM3, TM6, TM6B and TM8 are convenient balancers for the third chromosome. Balancer chromosomes can be obtained from The Bloomington Drosophila Stock Center at Indiana University. A complete list of available balancer chromosomes is available at http://www.flystocks.bio.indiana.edu. Methods of constructing stocks utilizing balancer chromosomes are well known in the art as described, for example, in Greenspan, supra, 1997.

Further provided by the invention is a method of identifying a therapeutic agent for treating Alzheimer's disease. The present invention also provides a method of identifying a therapeutic agent for treating Alzheimer's disease. The method includes the steps of (a) producing test progeny by performing matings between a first parent strain carrying a mutation in an Alzheimer's disease gene and a second parent strain containing a genetic variation where, in the absence of an agent, the parent strains produce test progeny having an altered phenotype relative to at least one sibling control; (b) administering an agent to the first or second parent strains or the test progeny; and (c) assaying the test progeny for the altered phenotype, where a modification of the altered phenotype producing a phenotype with more similarity to a wild type phenotype than the altered phenotype has to the wild type phenotype indicates that the agent is a therapeutic agent. An Alzheimer's disease gene useful for identifying a therapeutic agent in a method of the invention can be, for example, Appl or Psn, and an altered phenotype to be assayed can be, for example, increased or decreased viability. The screening assays disclosed herein are particularly useful in that they facilitate the analysis of randomly or rationally designed agents such as drugs, peptides, peptidomimetics, and the like to identify those agents that are therapeutic agents for treatment of Alzheimer's disease.

As used herein, the term “agent” means a biological or chemical compound such as a simple or complex organic molecule and is a molecule that, when administered, potentially produces a modification of an altered phenotype such as a complete or partial reversion of the phenotype. Such an agent can be, for example, a macromolecule, such as a small organic or inorganic molecule; a peptide including a variant or modified peptide or peptide mimetic; a protein or fragment thereof; an antibody or fragment thereof; a nucleic acid molecule such as a deoxyribonucleic or ribonucleic acid molecule; a carbohydrate; an oligosaccharide; a lipid, a glycolipid or lipoprotein, or any combination thereof. It is understood that an agent can be a naturally occurring or non-naturally occurring molecule such as a synthetic derivative, analog, or mimetic of a naturally occurring molecule.

If desired, an agent can be combined with, or dissolved in, a compound that facilitates uptake or delivery of the agent to a parent strain. Useful compounds include organic solvents such as dimethyl sulfoxide (DMSO) or ethanol; aqueous solvents such as water or physiologically buffered saline; and other solvents or vehicles such as glycols, glycerol, oils or organic esters. Such compounds, which can act, for example, to stabilize or to increase the absorption of the agent, include carbohydrates, such as glucose, sucrose or dextrans; antioxidants, such as ascorbic acid or glutathione; chelating agents; low molecular weight proteins; and other stabilizers or excipients. One skilled in the art would know that the choice of a carrier compound depends on the species of the parent strains and the route of administration.

In a method of the invention for identifying a therapeutic agent for treating Alzheimer's disease, test progeny are assayed for a modification of the altered phenotype such as a complete or partial reversion of the altered phenotype. As used herein, the term “therapeutic agent” means an agent that produces a modification of the altered phenotype which has more similarity to the wild type phenotype than the altered phenotype has to the wild type phenotype. Such a therapeutic agent is useful for ameliorating Alzheimer's disease in mammals such as humans. A therapeutic agent can reduce one or more symptoms of Alzheimer's disease, delay onset of one or more symptoms, or prevent or cure Alzheimer's disease.

In one embodiment, the therapeutic agent produces a complete or partial reversion of the altered phenotype. As used herein, the term “complete or partial reversion” means a decrease in or abolishment of an altered phenotype previously established to be associated with test progeny carrying a mutation in an Alzheimer's disease gene and a genetic variation. Thus, complete or partial reversion of an altered phenotype such as the decrease in viability of about 67.9% produced by the combination of Appl^{d} and Df(1) N8, can be, for example, an increase in viability of about 5%, 10%, 15%, 30%, 60%, or more.

A variety of modes of administration are contemplated for use in the methods of the invention depending, in part, on the species of the parent strains. In Drosophila, an agent can be administered, for example, in a 1% solution of sucrose and fed to a parent strain for an appropriate amount of time, for example, overnight. For administration to mice (M. musculus) or nematodes (C. elegans) the agent can be combined with solid food. For administration to a parent strain living in water such as zebrafish (D. rerio), an agent can be administered, for example, by adding it directly to the water. For administration to yeast (S. cerevisiae or S. pombe), an agent can be combined with solid support media such as agarose. Thus, one skilled in the art understands that the screening methods of the invention can be practiced using a variety of modes of administration including ingestion, injection, immersion or aerosol delivery using, for example, an atomizer or vaporization.

In the methods of the invention for identifying a therapeutic agent, an agent can be administered to one or both parent strains, to the test progeny, or to a combination thereof, before, during or after the mating. In one embodiment, the agent is administered to the first and second parent strains as well as to the test progeny. One skilled in the art understands that, where an agent is administered to only one parent strain, the agent is preferentially administered to females, which can carry either the mutation in the Alzheimer's disease gene or the genetic variation. Furthermore, it is understood that, where an agent is administered to test progeny, the agent can be administered at any stage of development including in utero; the timing of administration depends, in part, on the phenotype to be assayed.

One skilled in the art understands that an agent can be administered one time or repeatedly using a single dose or a range of doses. Appropriate concentrations of an agent to be administered in a method of the invention can be determined by those skilled in the art, and will depend on the chemical and biological properties of the agent, the mode of administration and the species of the parent strains. Exemplary concentration ranges to test in Drosophila are from about 1 mg/ml to 200 mg/ml of agent in, for example, sucrose solution, administered for a desired period of time.

In the methods of the invention for identifying a therapeutic agent for treating Alzheimer's disease, an agent can be administered individually, or a population of agents, which can be a small population or large diverse population, can be administered en masse. A population of agents, denoted a “library,” can contain, for example, more than 10; 20; 100; 10³, 10⁴, 10⁶, 10⁸, 10¹⁰, 10¹² or 10¹⁵ distinct agents. For example, test progeny produced from the mating of a parent Appl^{d} strain and a strain carrying the deficiency Df(1)RK2 are about 22.4% more viable than their control siblings (see Example I). A population of agents can be administered and the Appl^{d}+/+Df(1)RK2 test progeny assayed for a complete or partial reversion of the increased viability observed in the absence of the population of agents; an active population can be subdivided and the assay repeated in order to isolate a therapeutic agent from the population.

Methods are well known in the art for producing a variety of libraries to be used in a screening method of the invention, including libraries containing chemical or biological molecules such as simple or complex organic molecules, metal-containing compounds, peptides, proteins, peptidomimetics, glycoproteins, lipoproteins, antibodies, carbohydrates, nucleic acids, and the like. As indicated above, such libraries can contain a few or a large number of different agents, varying from about two to about 10¹⁵ agents or more. Furthermore, the chemical structure of the agents within a library can be related to each other or diverse. If desired, the agents constituting the library can be linked to a common or unique tag, which can facilitate recovery or identification of a therapeutic agent.

Libraries containing diverse populations of various types of peptide, peptoid and peptidomimetic agents can be routinely prepared by well known methods or obtained form commercial sources (see, for example, Ecker and Crooke, Biotechnology 13:351-360 (1995), and Blondelle et al., Trends Anal. Chem. 14:83-92 (1995), and the references cited therein, each of which is incorporated herein by reference; see, also, Goodman and Ro, Peptidomimetics for Drug Design, in “Burger's Medicinal Chemistry and Drug Discovery” Vol. 1 (ed. M. E. Wolff; John Wiley & Sons 1995), pages 803-861, and Gordon et al., J. Med. Chem. 37:1385-1401 (1994)). Where an agent is a peptide, protein or fragment thereof, the agent can be produced in vitro or can be expressed from a recombinant or synthetic nucleic acid molecule. Methods of synthetic peptide and nucleic acid chemistry are well known in the art.

A library of peptide agents also can be produced, for example, by constructing a cDNA expression library from mRNA collected from a cell, tissue, organ or organism of interest. Methods for producing such peptide libraries are well known in the art (see, for example, Ausebel et al. (Ed.), supra, 1989). Libraries of peptide agents also encompass those generated by phage display technology, which includes the expression of peptide molecules on the surface of phage as well as other methodologies by which a protein ligand is or can be associated with the nucleic acid molecule which encodes it. Methods for production of phage display libraries, including vectors and methods of diversifying the population of peptides which are expressed, are well known in the art (see, for example, Smith and Scott, Methods Enzymol. 217:228-257 (1993); Scott and Smith, Science 249:386-390 (1990); and Huse, WO 91/07141 and WO 91/07149).

A library of agents also can be a library of nucleic acid molecules, which can be DNA, RNA or analogs thereof. For example, a cDNA library can be constructed from mRNA collected from a cell, tissue, organ or organism of interest, or genomic DNA can be treated to produce appropriately sized fragments using restriction endonucleases or methods that randomly fragment genomic DNA. A library containing RNA molecules can be constructed, for example, by collecting RNA from cells or by synthesizing the RNA molecules chemically. Diverse libraries of nucleic acid molecules can be made using solid phase synthesis, which facilitates the production of randomized regions in the molecules. If desired, the randomization can be biased to produce a library of nucleic acid molecules containing particular percentages of one or more nucleotides at a position in the molecule (U.S. Pat. No. 5,270,163, issued Dec. 14, 1993).

Nucleic acid agents also can be nucleic acid analogs that are less susceptible to degradation by nucleases. RNA molecules containing 2′-O-methylpurine substitutions on the ribose residues and short phosphorothioate caps at the 3′- and 5′-ends, for example, exhibit enhanced resistance to nucleases (Green et al., Chem. Biol. 2:683-695 (1995)). Similarly, RNA containing 2′-amino-2′-deoxypyrimidines or 2′-fluro-2′-deoxypyrimidines is less susceptible to nuclease activity (Pagratis et al., Nature Biotechnol. 15:68-73 (1997)). Furthermore, L-RNA, which is a stereoisomer of naturally occurring D-RNA, is resistant to nuclease activity (Nolte et al., Nature Biotechnol. 14:1116-1119 (1996); and Klobmann et al., Nature Biotechnol. 14:1112-1115 (1996)). Such RNA molecules and routine methods of producing them are well known in the art (see, for example, Eaton and Piekern, Ann. Rev. Biochem. 64:837-863 (1995)). DNA molecules containing phosphorothioate linked oligodeoxynucleotides are nuclease resistant (Reed et al., Cancer Res. 50:6565-6570 (1990)). Phosphorothioate-3′ hydroxypropylamine modification of the phosphodiester bond also reduces the susceptibility of a DNA molecule to nuclease degradation (see Tam et al., Nucl. Acids Res. 22:977-986 (1994)). If desired, the diversity of a DNA library can be enhanced by replacing thymidine with 5-(1-pentynyl)-2′-deoxoridine (Latham et al., Nucl. Acids Res. 22:2817-2822 (1994)).

A library of agents also can be a library of chemical compounds, which can be generated, for example, by combinatorial chemical methods. Such a library can contain, for example, chemical oligomers such as peptoids, tertiary amines and ethylene glycols; decorated monomers such as benzodiazapines, sugar analogs, β-mercaptoketones and aminimides; and modified biological monomers such as sugar derivatives and random chemistry monomers. Such a library also can contain, for example, biological oligomers such as peptides, oligonucleotides, oligosaccharides, polysomes, random chemistry oligomers, phage proteins and bacterial membrane proteins. Applications of combinatorial technologies to drug discovery and library screening strategies are well known in the art as described, for example, in Gordon et al., supra, 1994; Ecker and Crooke, supra, 1995), and Jung, Combinatorial Peptide and Nonpeptide Libraries: A Handbook, VCH Publishers, Weinheim (FRG) and New York, N.Y. (USA)(1996). Combinatorial chemical libraries for screening also can also be obtained commercially, for example, from Trega Biosciences Inc. (San Diego, Calif.); ProtoGene Inc. (Palo Alto, Calif.); Array Biopharma Inc. (Boulder, Colo.); or Maxygen Inc. (Redwood City, Calif.).

As disclosed herein, a complementary molecular analysis was performed to assay for differential expression of mRNA and protein levels in Appl^{d} flies compared to Appl⁺ flies. Using differential display analysis, DNA microarrays or 2D gel electrophoresis coupled with mass spectrophotometry, a variety of mRNAs or proteins were isolated that are differentially expressed in Appl^{d} versus Appl⁺ D. melanogaster. Each of these differentially expressed mRNAs or proteins can correspond to a gene that is a member of an Alzheimer's disease genetic network and, thus, can correspond to an Alzheimer's disease gene useful in the above methods for identifying a therapeutic agent for treating Alzheimer's disease.

Thus, the invention provides an isolated nucleic acid molecule which is differentially expressed in Appl^{d} versus Appl⁺ D. melanogaster and contains a nucleic acid sequence having substantially the sequence of one of SEQ ID NOS: 1 to 63. Such a differentially expressed nucleic acid molecule can have, for example, the sequence of one of SEQ ID NOS: 1 to 63. Also provided herein is an isolated nucleotide sequence that contains at least 10 contiguous nucleotides of the nucleic acid sequence of one of SEQ ID NOS: 1 to 63.

The invention also provides an isolated nucleic acid molecule which is differentially expressed in Appl^{d} versus Appl⁺ D. melanogaster and contains a nucleic acid sequence having substantially the sequence of one of SEQ ID NOS: 64 to 80. Such an isolated nucleotide sequence can have, for example, the sequence of one of SEQ ID NOS: 64 to 80. The invention additionally provides an isolated nucleotide sequence containing at least 10 contiguous nucleotides of the nucleic acid sequence of one of SEQ ID NOS: 64 to 80.

As disclosed herein in Example II, differential display and DNA microarray analyses were used to identify more than 80 differentially expressed nucleic acid molecules (SEQ ID NOS: 1 to 80). Using differential display, 17 transcripts were increased while 46 transcripts were decreased in Appl^{d} flies relative to Appl⁺ flies. In particular, transcripts 27.1 (SEQ ID NO: 20); 27.1a (SEQ ID NO: 21); 27.4 (SEQ ID NO: 23); 27.5 (SEQ ID NO: 25); 27.5b (SEQ ID NO: 26); 30.3 (SEQ ID NO: 36); 30.3b (SEQ ID NO: 37); 30.9 (SEQ ID NO: 42); 30.9b (SEQ ID NO: 43); 48.1 (SEQ ID NO: 51); 48.1b (SEQ ID NO: 52); 48.2 (SEQ ID NO: 53); 48.3 (SEQ ID NO: 54); 49.3 (SEQ ID NO: 55); 58.1 (SEQ ID NO: 56); 58.4 (SEQ ID NO: 60); and 58.4a (SEQ ID NO: 61) were increased in Appl^{d} flies relative to Appl⁺ flies. Furthermore, transcripts A1 (SEQ ID NO: 1); 22.1 (SEQ ID NO: 2); 22.2 (SEQ ID NO: 3); 23.1 (SEQ ID NO: 4); 23.1b (SEQ ID NO: 5); 23.4 (SEQ ID NO: 6); 23.5 (SEQ ID NO: 7); 23.6 (SEQ ID NO: 8); 23.7 (SEQ ID NO: 9); 23.7b (SEQ ID NO: 10); 24.1 (SEQ ID NO: 11); 24.3 (SEQ ID NO: 12); 24.3a (SEQ ID NO: 13); 24.4 (SEQ ID NO: 14); 24.5 (SEQ ID NO: 15); 25.1 (SEQ ID NO: 16); 25.1b (SEQ ID NO: 17); 26.1 (SEQ ID NO: 18); 26.3 (SEQ ID NO: 19); 27.2 (SEQ ID NO: 22); 27.4b (SEQ ID NO: 24); 27.5c (SEQ ID NO: 27); 27.15 (SEQ ID NO: 28); 27.18 (SEQ ID NO: 29); 28.2 (SEQ ID NO: 30); 28.3 (SEQ ID NO: 31); 29.3 (SEQ ID NO: 32); 29.3b (SEQ ID NO: 33); 29.4 (SEQ ID NO: 34); 30.1 (SEQ ID NO: 35); 30.4 (SEQ ID NO: 38); 30.4b (SEQ ID NO: 39); 30.7 (SEQ ID NO: 40); 30.7b (SEQ ID NO: 41); 30.12 (SEQ ID NO: 44); 30.12b (SEQ ID NO: 45); 34.2 (SEQ ID NO: 46); 35.1 (SEQ ID NO: 47); 35.2 (SEQ ID NO: 48); 37.9 (SEQ ID NO: 49); 45.1 (SEQ ID NO: 50); 58.2 (SEQ ID NO: 57); 58.2b (SEQ ID NO: 58); 58.3 (SEQ ID NO: 59); and 60.2 (SEQ ID NO: 62) were decreased in Appl^{d} flies as compared to Appl⁺ flies.

Differential expression also was observed for 45 mRNAs using a DNA microarray made from 400 randomly chosen ESTs from the Berkeley Drosophila Genome Project UniGene Library. Of these differentially expressed mRNAs, 24 had increased expression and 21 had decreased expression in Appl^{d} relative to Appl⁺. Among these mRNAs, GH03592 (SEQ ID NO: 64); GH03824 (SEQ ID NO: 65); GH01554 (SEQ ID NO: 66); GH01770 (SEQ ID NO: 67); GH01730 (SEQ ID NO: 68); GH01988 (SEQ ID NO: 69); GH01718 (SEQ ID NO: 70); GH01072 (SEQ ID NO: 71); GH03622 (SEQ ID NO: 72); GH01420 (SEQ ID NO: 73); GH05210 (SEQ ID NO: 74); GH01717 (SEQ ID NO: 75); and GH01942 (SEQ ID NO: 76) exhibited increased expression in Appl^{d} relative to Appl⁺. In contrast, GH04745 (SEQ ID NO: 77); GH04984 (SEQ ID NO: 78); GH04859 (SEQ ID NO: 79); and GH03649 (SEQ ID NO: 80) had decreased expression in Appl^{d} relative to Appl⁺.

In addition, several differentially expressed mRNAs were identified as known genes: kismet (kis), a gene encoding a chromatin factor that interacts with Notch (Daubresse et al., Development 126:1175-1187 (1999)), and mitochondrial processing peptidase beta-subunit/vesicle trafficking protein SEC22B (Paces et al., Proc. Natl. Acad. Sci. USA 90:5355-5358 (1993); Mao et al., Proc. Natl. Acad. Sci. USA 95:8175-8180 (1998)) were increased in Appl^{d} relative to Appl⁺. Frequenin, encoding a calcium-sensitive-guanyl-cyclase-activator (Pongs et al., Neuron 11:15-28 (1993)); myosin-IB (Myo61F, Morgan et al., J. Mol. Biol. 239:347-356 (1994)); leonardo (leo), encoding a 14-3-3ζ protein (Skoulakis and Davis, Neuron 17:931-944 (1996)); and fly homologs of the mammalian phosphatidic acid phosphatase 2a2 (Leung et al., DNA Cell Biol. 17:377-385 (1998)) exhibited decreased expression in Appl^{d} relative to Appl⁺.

As further disclosed herein, several genes with an altered expression level in Appl^{d} flies were analyzed phenotypically for an interaction with the Appl network. After crossing the fly mutants with Appl^{d} flies, the various progeny classes were scored for viability as described above. Two temperature-sensitive alleles of the dynamin-encoding shibire locus were tested with Appl^{d}, and both showed reductions in viability that were temperature-sensitive (see Table 3). α-adaptin (Dornan et al., Mol. Biol. Cell 8:1391-1403 (1997)) and the fly's δ-adaptin homolog, garnet (Ooi et al., EMBO J. 16:4508-4518 (1997)) also were tested for an effect on viability when combined with Appl^{d}. These mutants affect genes whose products are involved in the same endocytic processes as dynamin. As shown in Table 3, the α-adaptin⁰⁶⁶⁹⁴ mutation increased viability in combination with Appl^{d}, whereas the garnet (g³) mutation in δ-adaptin decreased viability, indicating that these genes may belong to a local network. Table 3 also shows that the cnc⁰⁹³²¹ mutation in the locus of cap-n-collar (cnc), a gene encoding a transcription factor with bZIP homology, affects viability in flies carrying no functional copy of Appl.

TABLE 3
Viability Tests of Appl^{d} with Other Loci

| | | | % | | % |
|---|---|---|---|---|---|
| | | | viability¹ | | viability² |
| | | | female | | male |
| | | | progeny | | progeny |
| | | | (1 dose | | (0 dose of |
| Male | x Female | N | Appl⁺) | N | Appl⁺) |
| w | w Appl^{d}/FM7 | 102 | 92.16 | | |
| shi^{tsl} | w Appl^{d}/FM7 20° C. | 126 | 93.85 | | |
| shi^{tsl} | w Appl^{d}/FM7 27° C. | 198 | 70.69* | | |
| shi^{tsl39} | w Appl^{d}/FM7 20° C. | 187 | 88.41 | | |
| shi^{tsl39} | w Appl^{d}/FM7 | 52 | 52.94* | | |
| sca¹ | w Appl^{d}/FM7 | 75 | 97.37 | | |
| na | w Appl^{d}/FM7 | 87 | 112.20 | | |
| har38 | w Appl^{d}/FM7 | 237 | 134.65* | | |
| g³ bw | w Appl^{d}/FM7 | 225 | 69.17** | | |
| eag¹ | w Appl^{d}/FM7 | 91 | 93.62 | | |
| rut²⁰⁵⁰ | w Appl^{d}/FM7 | 147 | 107.04 | | |
| amn^{28A} | w Appl^{d}/FM7 | 87 | 97.73 | | |
| hsCreb17-2 | w Appl^{d}/FM7 27° C. | 87 | 112.20 | 72 | 414.29** |
| hsCrebC28 | w Appl^{d}/FM7 27° C. | 83 | 102.44 | 55 | 57.14* |
| cnc⁰⁹³²¹/ | w Appl^{d}/FM7 | 103 | 94.33 | 67 | 48.89** |
| CyO | | | | | |
| Su(H)I | w Appl^{d}/FM7 | 101 | 62.90* | | |
| Dl⁷/TM3 | w Appl^{d}/FM7 | 85 | 296.36** | | |
| α-adaptin/ | w Appl^{d}/FM7 | 92 | 268.00** | | |
| CyO | | | | | |
| w Appl^{d} | stnA¹⁵/FM7 | 145 | 93.33 | | |
| w Appl^{d} | wN²⁶⁴⁻³⁹/FM7 | 519 | 43.77* | | |
| shi^{tsl39} | wN²⁶⁴⁻³⁹/FM7 18° C. | 68 | 44.68** | | |
| w Appl^{d} | bib¹/CyO | 142 | 65.11* | | |
| w Appl^{d} | mam⁸/CyO | 36 | 381.82** | | |
| w Appl^{d} | Dhod⁸/TM3 | 42 | 90.91 | | |
| w Appl^{d} | Psn^{B3}/TM3 | 76 | 111.11 | | |
| w Appl^{d} | CrebA⁰³⁵⁷⁶/ | 90 | 177.78* | | |
| | TM3 | | | | |
| hsCreb17-2 | α-adaptin/ | 57 | 185.00* | | |
| | CyO | | | | |

¹for crosses 1-21, % viability = [(# mutant/w Appl^{d})/(#FM7/w Appl^{d})] × 100
for crosses 22-26, % viability = [(# w Appl^{d}/+; mutant/+)/(#w Appl^{d}/+; Balancer/+)] × 100
²% viability = [(# w Appl^{d}/Y; mutant/+)/(#w Appl^{d}/Y; Balancer/+)] × 100
*indicates significant departure (P < 0.05) from 100% by χ² test
**indicates significant departure (P < 0.01) from 100% by χ² test
All crosses carried out at 25° C. unless otherwise indicated

TABLE 4
Gene Expression Differences

| | |
|---|---|
| Decreased expression in Appl^{d} | Increased expression in Appl^{d} |

mRNA

| | |
|---|---|
| cap-n-collar (cnc) | kismet (kis) |
| tat-binding protein-1 (Tbp-1) | Frequenin (Frq) |
| shibire (shi) | ribosomal protein L9 (RpL9) |
| 14-3-3ζ (leo) | pheromone-binding protein- |
| | related protein 2 |
| | (Pbprp2) |
| dihydroorotate dehydrogenase | exocyst protein 84^{r} |
| (Dhod) | |
| myosin-IB (Myo61F) | mitochondrial processing |
| | peptidase-β/vesicle |
| | trafficking protein SEC22B^{h} |
| mitochondrial aldehyde | |
| dehydrogenase^{m} | |

Protein

| | |
|---|---|
| numb | adenyl cyclase (Ac39E) |
| hunchback (hb) | |
| α-actinin (Actn) | |
| actin57B (Act57B) | |
| mutant nucleotide excision | |
| repair protein (mus201) | |

^{r}rat homolog
^{m}mouse homolog
^{h}human homolog

TABLE 5
Identified Genes Differing between Appl^{d} and Appl⁺

| Gene | Fly | Human |
|---|---|---|
| Appl | J04516 | Y00264 |
| Notch | M16150/M11664 | M73980 |
| Delta | X06289 | X80903 (mouse) |
| big brain | X53275 | U36308 |
| mastermind | X54251 | — |
| Suppressor of Hairless | M94383 | L07872 |
| CrebA | M87038 | AF009368 |
| CrebB (inhibitor) | S79274 | NM_001881 |
| CrebB (activator) | S79274 | NM_004379 |
| cap-n-collar | M37495 | X77366 |
| kismet | AF113847 | — |
| tat-binding protein-1 (Tbp-1) | AF134402 | M34079 |
| Frequenin | L08064 | S47565 |
| shibire | X59435 | NM_004408 |
| α-adaptin | Y13092 | AF049527 |
| δ-adaptin | AF002164 | AF002163 |
| ribosomal protein L9 | X94613 | NM_000661 |
| 14-3-3ζ | Y12573 | MM_003406 |
| pheromone-binding | U05981 | — |
| protein-related protein-2 | | |
| dihydroorotate dehydrogenase | L00964 | M94065 |
| exocyst protein 84 | — | AF032669 |
| myosin-IB | U07596 | U57053 |
| mitochondrial processing | — | NM_004279 |
| peptidase-β | | |
| vesicle trafficking protein | — | NM_004892 |
| (SEC22B) | | |
| phosphatidic acid phosphatase | — | AF014403 |
| 2a2 | | |
| mitochondrial aldehyde | — | NM_000690 |
| dehydrogenase | | |
| numb | M27815 | NM_003744 |
| adenyl cyclase | AF005629 | U65474 |
| hunchback | U17742 | — |
| α-actinin | X51753 | D89980 |
| actin57B | K00673 | X04098 |
| mutant nucleotide excision | AF162795 | X69978 |
| repair protein | | |
| (mus201)/human xeroderma | | |
| pigmentosum VII | | |

The term “isolated,” as used herein in reference to a nucleic acid molecule, nucleotide sequence or protein of the invention, means a nucleic acid molecule, nucleotide sequence or protein that is in a form relatively free from contaminating lipids, unrelated nucleic acids, unrelated proteins and other cellular material normally associated with a nucleic acid molecule or protein in a cell.

As used herein, the term “nucleic acid molecule” means a deoxyribonucleic acid (DNA) or ribonucleic acid (RNA) molecule that can optionally include one or more non-native nucleotides, having, for example, one or more modifications to the base, sugar, or phosphate portion, or can include a modified phosphodiester linkage. The term nucleic acid molecule includes both single-stranded and double-stranded nucleic acid molecules, which can represent the sense strand, anti-sense strand, or both, and includes linear, circular and branched conformations. Exemplary nucleic acid molecules include genomic DNA, cDNA, mRNA and oligonucleotides, corresponding to either the coding or non-coding portion of the molecule. A nucleic acid molecule of the invention can additionally contain, if desired, a detectable moiety such as a radiolabel, fluorochrome, ferromagnetic substance, luminescent tag or a detectable agent such as biotin.

As used herein, the term “nucleotide sequence” means a single-stranded nucleic acid sequence that can range in size from about 10 contiguous nucleotides to the full-length of a nucleic acid molecule of the invention. A nucleotide sequence of the invention, which can be useful, for example, as a primer for PCR amplification, can have a sequence of at least, for example, 10, 11, 12, 13, 14, 15, 16, 17, 18, 19, 20, 25, 30, 35, 50, 100, 200, or more nucleotides.

The term “differentially expressed,” as used herein, means the increased or decreased expression of a nucleic acid molecule or protein in a first genetic background as compared to a second genetic background. In general, the term differentially expressed is used herein to refer to increased or decreased expression of a nucleic acid molecule or protein in a genetic background in which the level or activity of an Alzheimer's disease gene product is abnormal compared to a genetic background of wild type gene product activity, for example Appl^{d} versus Appl⁺.

The term “substantially the sequence,” as used herein in reference to a differentially expressed nucleic acid sequence of the invention, is intended to mean one of the sequences shown as SEQ ID NOS:l to 80, or a similar, non-identical sequence that is considered by those skilled in the art to be a functionally equivalent sequence. For example, a nucleic acid sequence that has one or more nucleotide additions, deletions or substitutions with respect to the indicated D. melanogaster nucleic acid sequence is encompassed by the invention, so long as the nucleic acid molecule retains its ability to selectively hybridize with the D. melanogaster nucleic acid sequence. A nucleic acid molecule having substantially the sequence of one of the indicated differentially expressed transcripts can be, for example, an isotype variant or species homolog, such as a vertebrate or invertebrate homolog, including a mammalian homolog such as murine, primate or human homolog.

The invention further provides an isolated protein which is differentially expressed in Appl^{d} versus Appl⁺ D. melanogaster and has a specific molecular weight and isoelectric point, or a homolog thereof. Thus, the invention provides, for example, an isolated A1.2 protein which is differentially expressed in Appl^{d} versus Appl⁺ D. melanogaster and has an approximate molecular weight of about 40 kDa and an approximate isoelectric point of 9.5, or a homolog thereof. The invention similarly provides the proteins A1.3 to A1.29 and the proteins W1.2 to W1.25, which have the approximate molecular weights and isoelectric points shown in Table 6. Molecular weights were determined by 2D gel electrophoresis (see Example IIC).

2D gel electrophoresis followed by in-gel trypsinization and MALDI-TOF analysis of the resulting tryptic fragments, was used to identify 54 protein differences, of which 29 represented increased protein levels and 25 represented decreased protein levels in Appl^{d} relative to Appl⁺.

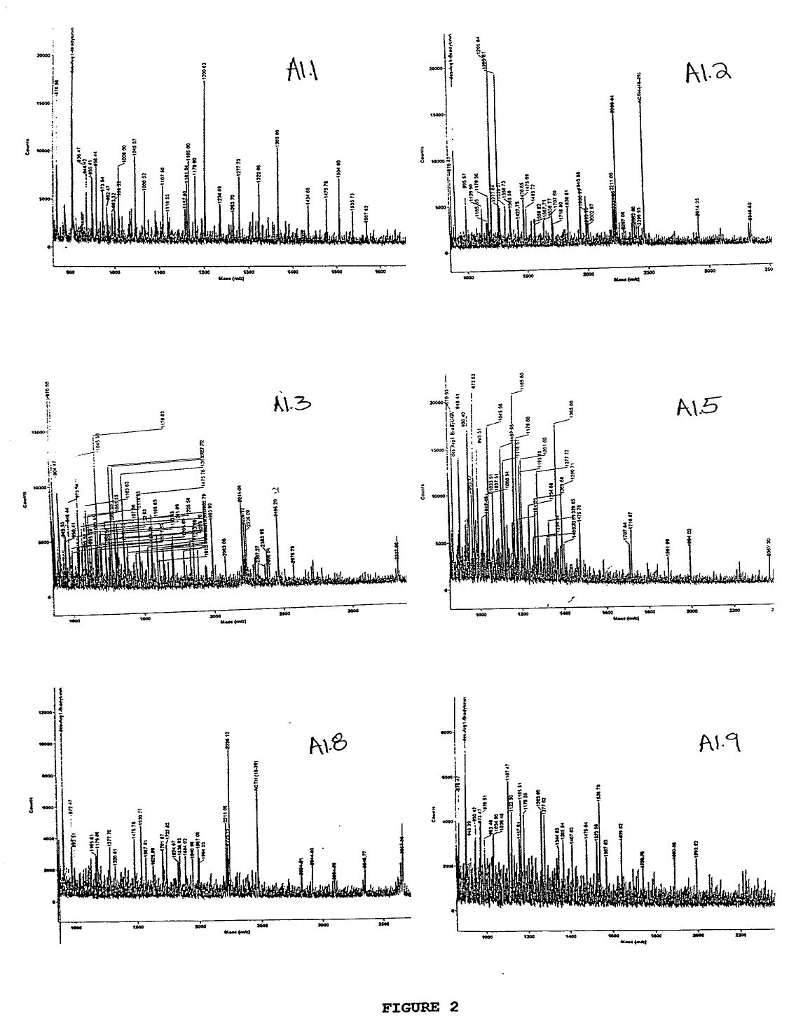

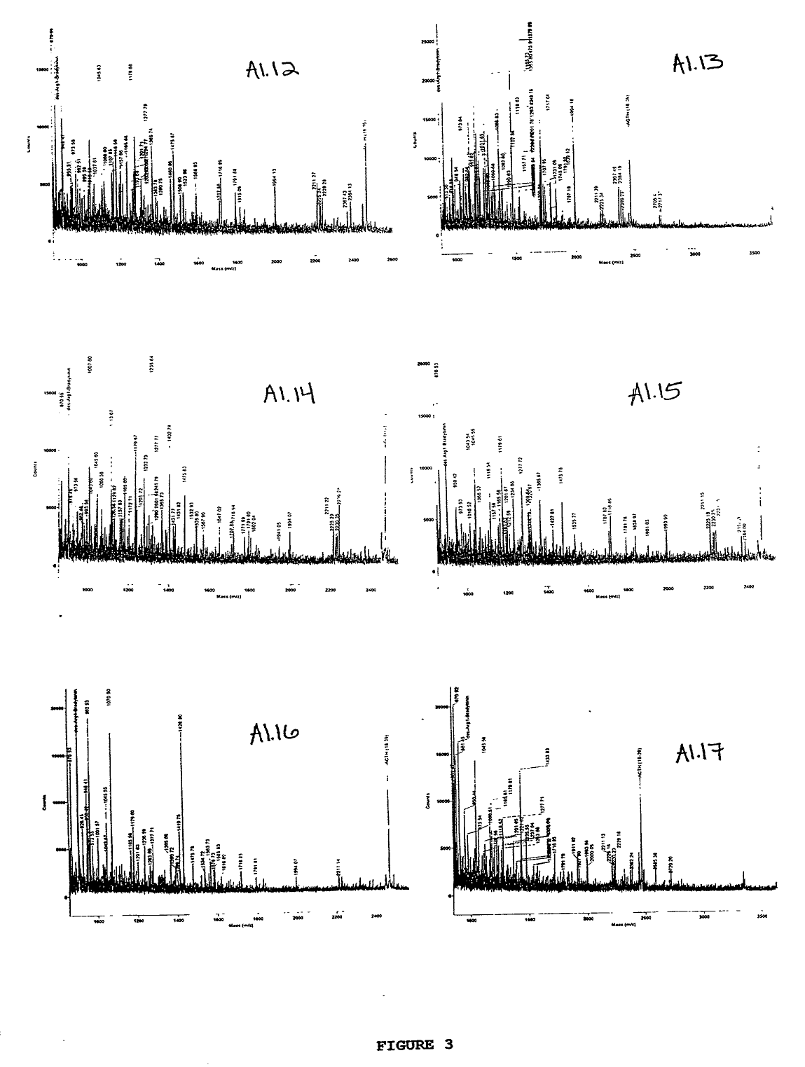

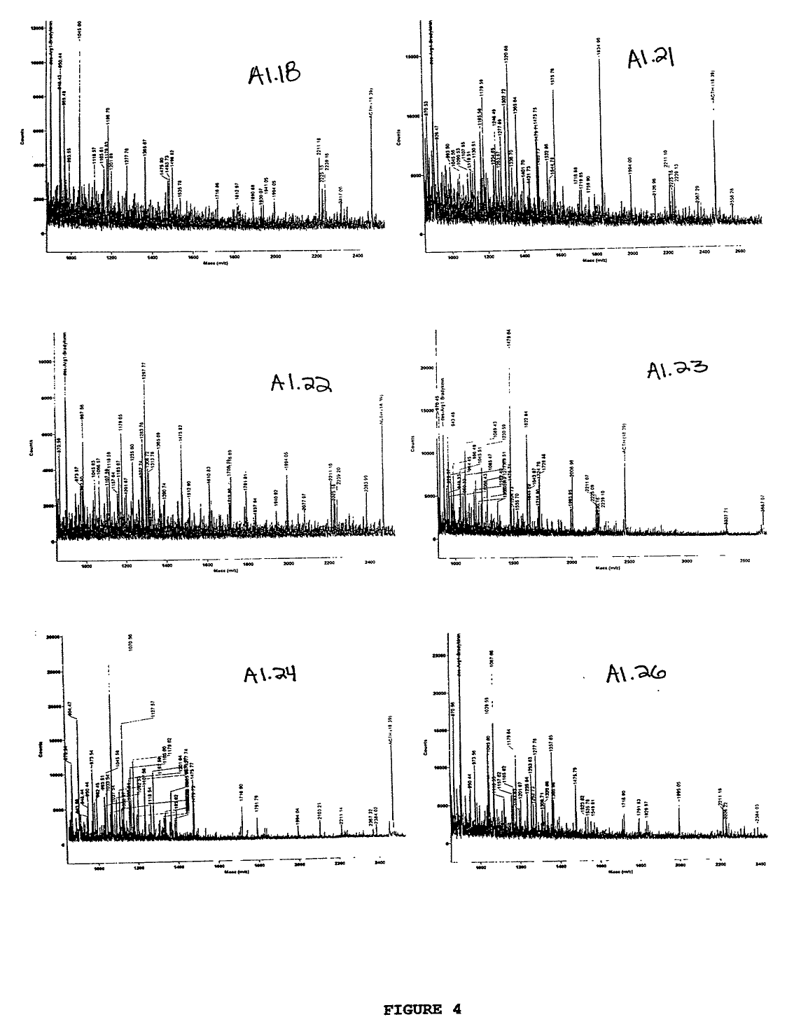

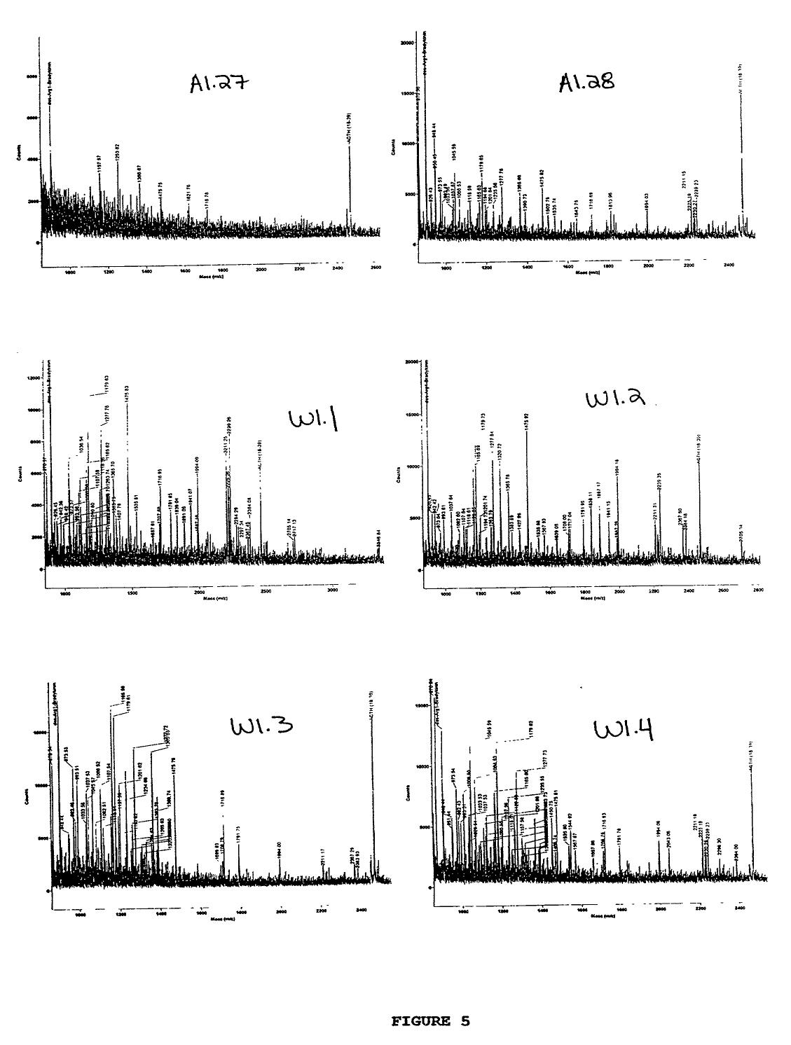

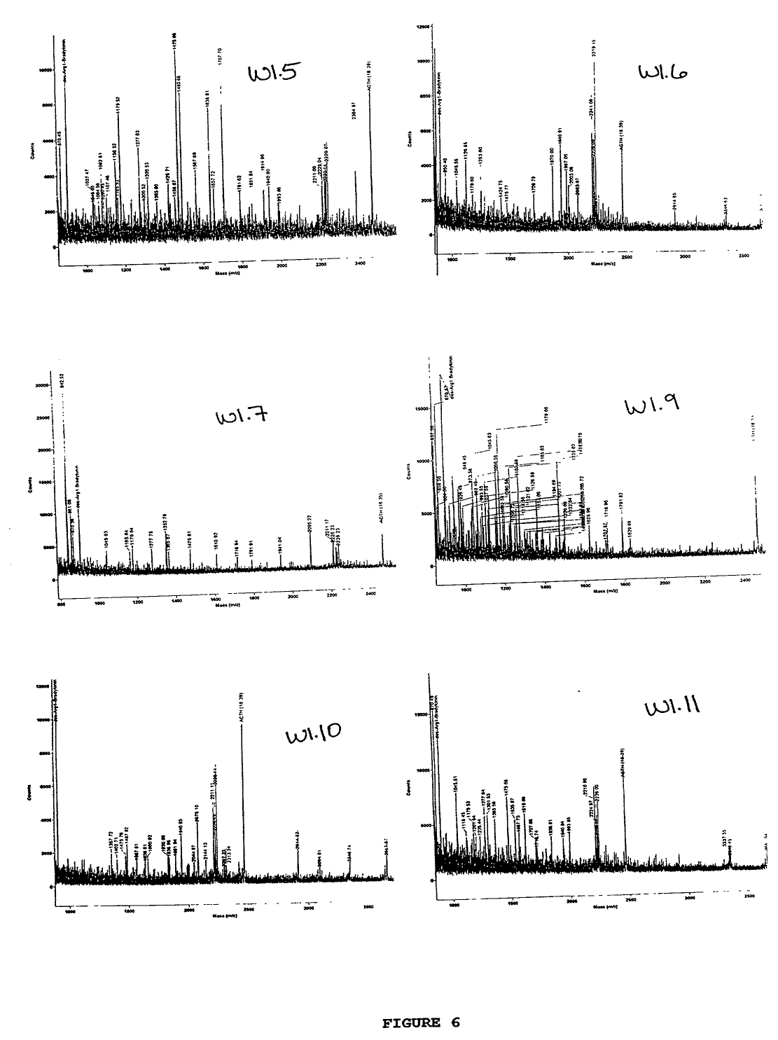

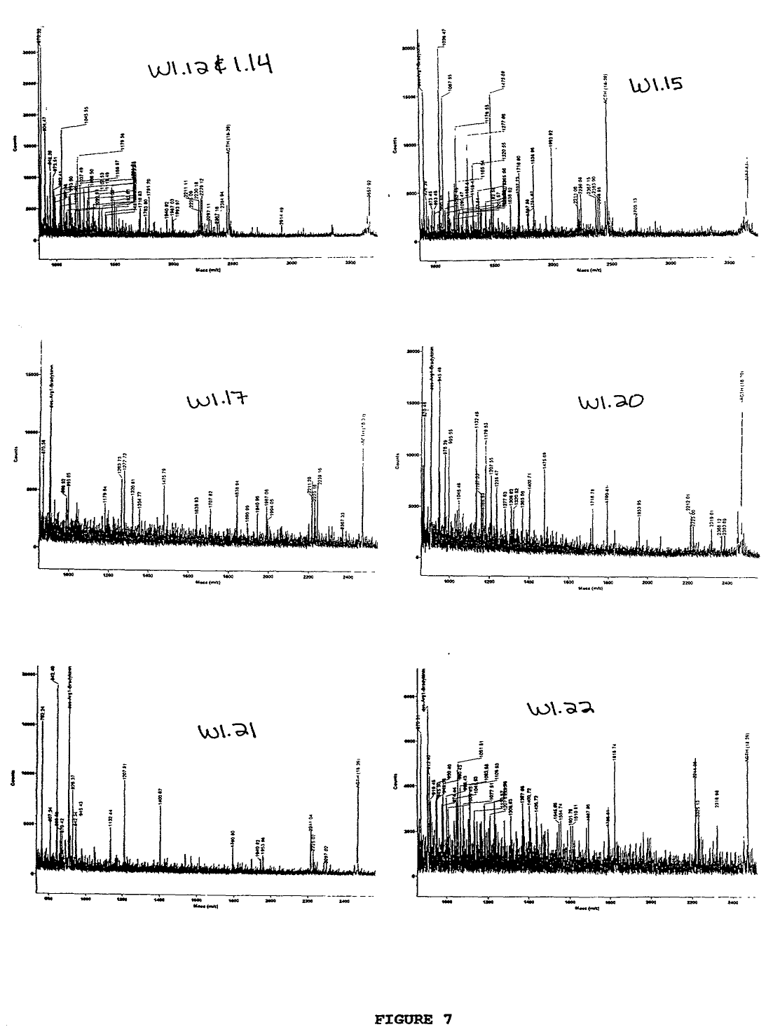

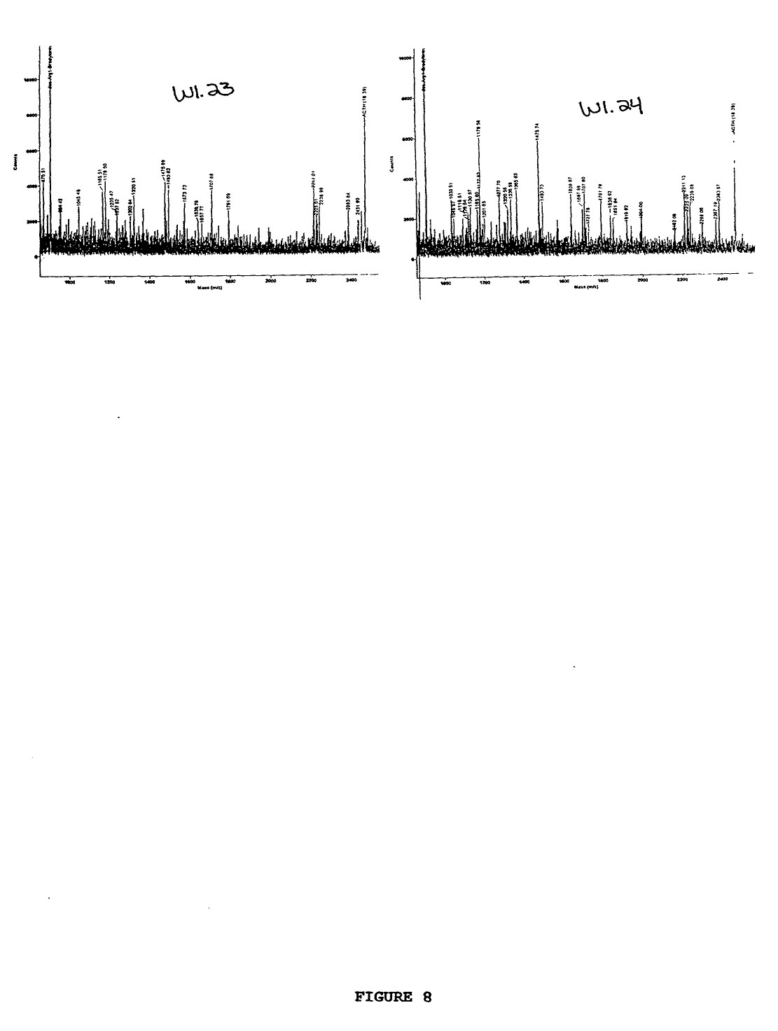

Proteins A1.2, A1.3, A1.4, A1.5, A1.6, A1.7, A1.8, A1.9, A1.10, A1.11, A1.12, A1.13, A1.14, A1.15, A1.16, A1.17, A1.18, A1.19, A1.20, A1.21, A1.22, A1.23, A1.24, A1.25, A1.26, A1.27, A1.28 and A1.29 are proteins with increased expression in w Appl^{d} as compared to w Appl⁺. W1.2, W1.4, W1.6, W1.7, W1.8, W1.9, W1.10, W1.12, W1.13, W1.14, W1.15, W1.16, W1.18, W1.19, W1.20, W1.22, W1.23, W1.24, and W1.25 are proteins with decreased expression in w Appl^{d} as compared to w Appl⁺. In addition to their differntial expression in Appl^{d} versus Appl⁺, these proteins are characterized by the molecular weights and isoelectric points shown in Table 6. Furthermore, these proteins are characterized by mass spectrophotometric analysis of in-gel tryptic digests, which are shown in FIGS. 2 to 8.

As shown in Table 4, five proteins with decreased expression in Appl^{d} flies were identified as follows: numb, a protein required for asymmetric cell divisions in neural development (Uemura et al., Cell 58:349-360 (1989)); hunchback, a transcription factor required for segmentation and for neural development (Tautz et al., Nature 327:383-389 (1987)); mus201, an excision repair protein (Houle and Friedberg, Gene 234:353-360 (1999)); and the cytoskeletal proteins α-actinin (Fyrberg et al., J. Cell Biol. 110:1999-2011 (1990)) and actin57B (Fyrberg et al., Cell 24:107-116 (1981)). One protein was identified with increased expression in Appl^{d} flies relative to Appl⁺ flies: an isoform of adenyl cyclase, Ac39E (Genbank AF005629), which is distinct from the one encoded by the rutabaga locus (see Table 4). Table 5 summarizes the genes identified as having differential mRNA or protein expression in Appl^{d} as compared to Appl⁺.

TABLE 6
Molecular Weights and Isoelectric Points to
Accompany Mass Spectra of Proteins Differing
on w Appl^{d} (A1) vs. w Appl⁺ (W1) 2D gels

| | MW (kD) | pI | | MW (kD) | pI |
|---|---|---|---|---|---|
| A1.2 | 32.1 | 9.5 | W1.2 | 19.1 | 7.7 |
| A1.3 | 47.9 | 9.5 | W1.4 | 15.7 | 9.0 |
| A1.4 | 10.9 | 8.4 | W1.6 | 27.4 | 7.4 |
| A1.5 | 13.9 | 5.8 | W1.7 | 27.4 | 8.0 |
| A1.6 | 21.6 | 6.6 | W1.8 | 33.4 | 6.7 |
| A1.7 | 42.5 | 6.7 | W1.9 | 51.9 | 6.8 |
| A1.8 | 47.9 | 6.7 | W1.10 | 60.9 | 6.8 |
| A1.9 | 51.9 | 6.7 | W1.12 | 23.3 | 6.8 |
| A1.10 | 80.6 | 6.8 | W1.13 | 22.4 | 6.9 |
| A1.11 | 28.5 | 6.2 | W1.14 | 22.4 | 7.0 |
| A1.12 | 32.1 | 6.3 | W1.15 | 9.3 | 6.8 |
| A1.13 | 30.9 | 6.7 | W1.16 | 11.4 | 6.8 |
| A1.14 | 37.7 | 6.8 | W1.18 | 12.3 | 6.4 |
| A1.15 | 33.4 | 6.8 | W1.19 | 9.7 | 6.4 |
| A1.16 | 68.7 | 5.8 | W1.20 | 42.5 | 4.4 |
| A1.17 | 66.0 | 6.4 | W1.22 | 56.2 | 4.5 |
| A1.18 | 80.6 | 6.7 | W1.23 | 26.3 | 4.4 |
| A1.19 | 98.4 | 6.2 | W1.24 | 24.3 | 4.5 |
| A1.20 | 110.9 | 5.9 | W1.25 | 22.4 | 5.6 |
| A1.21 | 44.2 | 5.7 | | | |
| A1.22 | 29.7 | 5.7 | | | |
| A1.23 | 23.3 | 5.6 | | | |
| A1.24 | 12.3 | 5.6 | | | |
| A1.26 | 10.5 | 5.6 | | | |
| A1.27 | 11.8 | 5.6 | | | |
| A1.28 | 18.4 | 5.5 | | | |
| A1.29 | 19.1 | 5.6 | | | |

Also encompassed by the invention are homologs of the differentially expressed D. melanogaster proteins having the characteristic molecular weights and isoelectric points shown in Table 6. Such homologs include vertebrate and invertebrate homologs, including mammalian homologs such as murine, primate and human homologs and generally share conserved sequence and function with the homologous D. melanogaster protein. A human or other homolog can share, for example, at least about 25%, 30%, 40%, 50%, 60%, 75%, 90% or 95% amino acid identity with the indicated D. melanogaster homolog. Such homologous proteins can be used, for example, to prepare antibodies; the homologous genes are useful, for example, as Alzheimer's disease genes in the screening methods of the invention described above.

### EXAMPLE I

### Gene-Network Mapping of Genes Acting in the Same Network as the Drosophila Amyloid Protein Precursor-Like Gene (Appl)

This example describes identification of genes acting within the same genetic network as Appl, the Drosophila homolog of human amyloid protein precursor (APP).

A. Interaction of Appl with X Chromosome Deficiencies

Male flies bearing a chromosome that lacks the Appl gene (w Appl^{d}; Kalpana White, Brandeis University, Waltham, Miss.), which is not required for viability or for most other functions in Drosophila (Luo et al., supra, 1992), were crossed with a series of FM7 virgin females bearing individual deficiencies of the X chromosome, “Df(1)s”. This set of deficiencies altogether covers roughly 70% of the X chromosome, nearly 15% of the entire genome and thus serves as a representative sample of the genome. In each case, the number and genotype of female adults emerging from each cross were scored, and the viability of the test genotype relative to sibling controls calculated (see Table 1). In 34 combinations of X chromosomal deficiencies with the Appl^{d} chromosome (Appl⁻/Df(1)), most had approximately normal viability, five had severely reduced viability (<35%), seven had moderately reduced viability, and three had increased viability relative to the control (Table 1).

Viability was determined as follows. Flies were cultured on brewer's yeast, dark corn syrup and agar food (modified from Bennett and van Dyke, Dros. Inform. Serv. 46:160 (1971)) at 25° C., 50-60% relative humidity and in 12 hr:12 hr light:dark cycles. Viability was scored by counting adult flies in the first or second day after emergence. Unless otherwise indicated, all fly stocks were obtained from the Bloomington Drosophila Stock Center.

B. Interaction of Appl^{d} with Network Candidate Genes

Four chromosome segments from the initial Appl^{d}/Df(1) screen described above were analyzed further.

As shown in Table 1, the deletion of segment 3C2;3E4, (Df(1)N8), when combined with Appl^{d}, resulted in progeny with 32% viability relative to the controls. This segment contains the Notch locus (Artavanis-Tsakonas et al., Science 284:770-776 (1999)), a human homolog of which has been implicated in a form of hereditary degenerative dementia (Joutel et al., supra, 1996). Notch is also known to interact genetically with Presenilin, the fly homolog of human Presenilin (Struhl and Greenwald, Nature 398:522-525 (1999); Ye et al., Nature 398:525-529 (1999)). A null allele of Notch, N²⁶⁴⁻³⁹, was tested in combination with Appl^{d} and found to result in a viability reduction similar to that of the deficiency Df(1)N8 (see Table 3).

Several mutants known to interact with Notch also were tested for their effects in the viability assay. Neither scabrous (sca), which encodes a protein with a fibrinogen-like beta and gamma chain C-terminal domain (Brand and Campos-Ortega, Roux Arch. Dev. Biol. 198:275-285 (1990)), nor Presenilin (Psn⁸³) mutations affected viability. Suppressor of Hairless (Su(H)1), which encodes a DNA-binding protein (Fortini and Artavanis-Tsakonas, Cell 79:273-282 (1994)), and big brain (bib), which encodes a channel-like transmembrane protein (Rao et al., Nature 345:163-167 (1990)), each gave moderate reductions in viability with Appl^{d}. Two other genes gave significant increases in relative viability when combined with Appl^{d}: mastermind (mam), a nuclear protein, (Smoller et al., Genes Dev. 4:1688-1700 (1990)) and Delta (Dl), a transmembrane protein that is a ligand of Notch (Vaessin et al., EMBO J. 6:3431-3440 (1987)). Notch and Delta gave reciprocal effects with Appl^{d}; these genes also are reciprocally regulated on cells as part of their normal signaling function (Heitzler and Simpson, supra, 1991).

The deletion of segment 17A1;18A2 (Df(1)N19) in combination with Appl^{d} resulted in 62.8% viability relative to sibling controls (Table 1). The 17A1;18A2 segment contains the dCrebB locus, a transcription factor implicated in neuronal plasticity and long-term memory formation (Dubnau and Tully, supra, 1998). The Appl^{d} chromosome was tested in combination with transgenic strains of Drosophila expressing either an activator form of dCrebB (C28) or an inhibitor form (17-2). As summarized in Table 3, the activator form produced a decrease in viability, whereas the inhibitor form produced an increase in viability relative to controls. These interactions were not apparent in flies carrying one dose of Appl⁺ (heterozygous females) but were evident in hemizygous males, which lack a functional copy of Appl⁺. The alterations in the viability seen with the Creb variants paralleled the reciprocity of cellular function for the two forms of the transcription factor. A mutation of the other dCreb locus on the third chromosome (CrebA⁰³⁵⁷⁶; Andrew et al., Development 124:181-193 (1997)), also was tested for the ability to interact with Appl^{d}. Increased viability was observed in flies carrying one dose of Appl⁺ (heterozygous females) and one dose of the hypomorphic CrebA⁰³⁵⁷⁶ mutation, as also observed for the dCrebB inhibitor.

The deletion of segment 12D2;13A5 (Df(1)RK2) in combination with Appl^{d} resulted in 122% viability relative to controls (Table 1). Of the several mutations from this region that are available, rutabaga (rut), encoding an adenyl cyclase involved in neuronal plasticity and learning (Livingstone et al., Cell 37:205-215 (1984)) and eag, encoding a potassium channel subunit (Warmke et al., Science 252:1560-1562 (1991)), gave no significant effects when combined with Appl^{d}. However, the halothane-resistant mutant (har38, Krishnan and Nash, Proc. Natl. Acad. Sci. USA 87:8632-8636 (1990)) resulted in increased viability in combination with Appl^{d}, consistent with the deficiency phenotype. har38 is allelic to narrow abdomen (na, Krishnan and Nash, supra, 1990), a morphological mutation, which showed no viability effect in combination with Appl^{d}. The allele-specific interaction indicates that har38 can interact directly with Appl in Drosophila. Given that halothane-sensitivity in flies is virtually identical to that of humans (Krishnan and Nash, supra, 1990), these results also indicate that the human homolog of har38 can interact with APP.

The deletion of a fourth segment, 18E-20 (Df(1)HF396), resulted in 11.9% viability relative to controls when combined with Appl^{d} (Table 1). Subdivision of the region into smaller segments showed no comparable reduction for any segments from 19A2 to 20EF. A mutant implicated in neuronal plasticity, amnesiac (amn^{28A}), was tested for interactions with Appl^{d}. This mutant, which is located at and encodes a PACAP-like neuropeptide required for the formation of medium-term memory, resulted in normal viability in combination with Appl^{d}, as did the stoned (stnA¹⁵) mutation at 20A4, which affects synaptic physiology and plasticity.

### EXAMPLE II

### Analysis of Differences in Gene Expression in Appl^{d} Versus Wild Type Flies

This example demonstrates the use of differential display, DNA microarray analysis and 2D gel electrophoresis to identify genes involved in an Alzheimer's disease gene network.

A. Differential Display Analysis

Differential display analysis of RNA isolated from adult heads of w Appl^{d} vs. wild type (w) siblings was used to identify genes with increased or decreased expression in Appl^{d}. Briefly, 20 fly heads were homogenized in TRIzol (Life Technologies, Inc., Frederick, Md.) and extracted according to the manufacturer's instructions. Differential display of mRNA was performed essentially as described in Cirelli and Tononi, Mol. Brain Res. 56:293-305 (1998). About 38 transcripts were identified with expression levels altered in Appl^{d}flies. Of these, 16 had increased expression, and 22 had decreased expression in Appl^{d} flies relative to Appl⁺ flies.

Sequence analysis using the BLAST program at either the Berkeley Drosophila Genome Project <http://www.fruitfly.org/blast/> or NCBI <http://www.ncbi.nlm.nih.gov/BLAST/> revealed that about 15% of the transcripts represented sequences currently available in Drosophila databases (Table 4). In particular, the following genes were identified with altered RNA expression levels in Appl^{d}flies. Shibire (shi), which encodes dynamin (van der Bliek and Meyerowitz, Nature 351:411-414 (1991)); cap-n-collar (cnc), which encodes a bZIP-like transcription factor (Mohler et al., Mech. Dev. 34:3-9 (1991)), Pbprp-2, which encodes a pheromone-binding-protein-related-protein (Pikielny et al., Neuron 12:35-49 (1994)); RpL9, encoding ribosomal protein L9 (Schmidt et al., Mol. Gen. Genet. 251:381-387 (1996)); Dhod (Jones et al., Mol. Gen. Genet. 219:397-403 (1989)), 18S ribosomal RNA; Tat-binding protein-1 (Tbp-1, FlyBase FBgn0026321) of the proteasome; and a homolog of rat exo84 of the exocyst secretion complex (Kee et al., Proc. Natl. Acad. Sci. USA 94:14438-14443 (1997)).

B. DNA Microarray Analysis

mRNA levels also were compared using a DNA microarray made from 400 randomly chosen ESTs from the Berkeley Drosophila Genome Project UniGene Library (http://www.fruitfly.org/EST/). Briefly, a glass slide DNA microarray was prepared as described in White et al., Science 286:2179-2184 (1999), from the UniGene Library (plates #54, 55, 56 and 57; Research Genetics, Huntsville, Ala.). PolyA+ RNA was prepared from groups of 100 whole flies using MicroPoly(A)Pure (Ambion, Austin, Tex.) according to the manufacturer's instructions, and subsequently labeled and hybridized to the arrays as described in White et al., supra, 1999. ESTs showing >2-fold expression difference were analyzed as described above.

Differential expression was observed for 45 mRNAs (24 increased and 21 decreased in Appl^{d} relative to Appl⁺). Of the 45 mRNAs, six were identified as known genes as follows: kismet (kis), a gene encoding a chromatin factor that interacts with Notch (Daubresse et al., supra, 1999); Frequenin, encoding a calcium-sensitive-guanyl-cyclase-activator (Pongs et al., supra, 1993); leonardo (leo), encoding a 14-3-3ζ protein (Skoulakis and Davis, supra, 1996); myosin-IB (Myo61F, Morgan et al., supra, 1994); fly homologs of the mammalian phosphatidic acid phosphatase 2a2 (Leung et al., supra, 1998) and mitochondrial processing peptidase beta-subunit/vesicle trafficking protein SEC22B (Paces et al., supra, 1993; Mao et al., supra, 1998).

C. 2D Cel Electrophoretic Analysis of Differentially Expressed Proteins

2D gel electrophoresis/mass spectrophotometry analysis was performed on adult head extracts from w Appl^{d} vs. w siblings (Gygi et al., Mol. Cell. Biol. 19:1720-1730 (1999)). Briefly, 35 fly heads were homogenized using the extraction protocol described in Unlu et al., Electrophoresis 18:2071-2077 (1997), and 2D gel electrophoresis was performed as follows. For the first dimension, samples were diluted up to 125 μl with a rehydration solution consisting of 8 M urea, 2 M thiourea, 2% CHAPS, 0.5% 3-10L IPG Buffer, and a trace of bromophenol blue. The samples were allowed to sit in the rehydration solution for 30 minutes before being applied to 3-10L Immobiline DryStrips. After rehydrating the strips for 12 hours at 20° C., they were electrophoresed on the Pharmacia IPGPhor in steps of 250Vh, 500Vh and 8000Vh. For the second dimension, the strips were equilibrated for 15 minutes in 5 mls of an equilibration solution consisting of 50 mM Tris-Cl pH 8.8, 6 M urea, 30% glycerol, 2% SDS, and 50 mgs of dithiothreitol. The gels were then overlayed on 4-20% pre-cast Biorad Mini-gels and electrophoresed for 33 minutes at 200V using standard SDS running buffer. Gels were silver stained using the Pharmacia Silver Staining Kit (Amersham-Pharmacia Biotech, Inc., Piscataway, N.J.). Molecular weights were approximated to +/−25% of the actual molecular weight using the following molecular weight standards 200 kD, 135 kD, 81 kD, 41.9 kD, 31.4 kD, 18 kD, and 6.9 kD.

Spots were matched between gels using the PDQuest program (BioRad, Hercules, Calif.), and those showing the strongest differences were then excised with a scalpel and subjected to in-gel trypsin digestion, extraction and purification in preparation for MALDI-TOF analysis (Helmann et al, Anal. Biochem. 224:451-455, (1995)), which was performed at the Scripps Research Institute Mass Spec Facility. The resulting spectra were analyzed by matching peptide patterns to those in the database (http://prowl.rockefeller.edu/cgi-bin/ProFound).

Using the above procedure, fifty-four protein differences were analyzed, of which 29 represented increased protein levels and 25 represented decreased protein levels in Appl^{d} relative to Appl⁺. Table 6 shows the molecular weights and isoelectric points of proteins differing in expression between Appl^{d} and Appl⁺.

Of the fifty-four protein differences analyzed, five proteins were identified as gene products of the following loci: numb, a protein required for asymmetric cell divisions in neural development (Uemura et al., Cell 58:349-360 (1989)); hunchback, a transcription factor required for segmentation and for neural development (Tautz et al., supra, 1987); mus201, an excision repair protein (Houle and Friedberg, supra, 1999); and the cytoskeletal proteins α-actinin (Fyrberg et al., supra, 1990) and actin57B (Fyrberg et al., supra, 1981). As shown in Table 4, one protein was elevated in Appl^{d} flies relative to Appl flies: an isoform of adenyl cyclase, Ac39E (Genbank AF005629), which is distinct from the one encoded by the rutabaga locus. Table 5 summarizes the genes identified as differing in expression between Appl^{d} and Appl⁺.

### EXAMPLE III

### Genetic and Behavioral Analysis of Loci with Differing Expression in Appl^{d} Flies

This example describes the genetic analysis of loci that exhibit altered expression levels in Appl^{d}.

A. Genetic Analysis of Genes Identified by Expression Analysis

Genes with an altered expression level in Appl^{d} flies were analyzed phenotypically for an interaction with the Appl network. Available mutants for the genes identified by expression analysis were obtained and crossed with Appl^{d} flies. The various progeny classes were scored for viability as described above.

The dynamin-encoding shibire locus is on the X chromosome at salivary chromosome band 14A1. A chromosomal deficiency including 14A1, Df(1)sd72b, showed a moderate decrease in viability with Appl^{d} (see Table 1). Two temperature-sensitive alleles of shibire were tested with Appl^{d}, and both showed reductions in viability that were temperature-sensitive (see Table 3).

Two other mutants in genes whose products are involved in the same endocytic processes as dynamin were tested for interactions with Appl^{d}: α-adaptin (Dornan et al., supra, 1997) and the fly's δ-adaptin homolog, garnet (Ooi et al., supra, (1997). The α-adaptin⁰⁶⁶⁹⁴ mutation increased viability in combination with Appl^{d} whereas the garnet (g³) mutation in δ-adaptin decreased viability (Table 3), indicating that these genes may belong to a local network. Although no fly mutant exists for exo84, a protein component of the exocyst complex involved in vesicle fusion and secretion in yeast and mammals (Kee et al., supra, 1997), this gene, which is differentially increased in Appl^{d}, also can be involved in the Appl network, given the phenotypic involvement of dynamin, α-adaptin and δ-adaptin. The cnc⁰⁹³²¹ mutation in the locus of cap-n-collar (cnc), a gene encoding a transcription factor with bZIP homology, affects viability in flies carrying no functional copy of Appl (see Table 3).

B. Analysis of Genetic Interactions Between Genes Affecting Appl

Notch and shibire, which share a common interaction with Appl^{d}, were analyzed further. When mutations in Notch (N²⁶⁴⁻³⁹) and shibire (shi^{st139}) were combined, the result was a major reduction in viability. Of those flies that did survive to adulthood, emergence was significantly delayed and the notched wing phenotype resulting from the combination of N²⁶⁴⁻³⁹ and shiₛₜ₁₃₉ was much more severe than for N²⁶⁴⁻³⁹ alone.

Possible lateral interactions were further analyzed between α-adaptin and the dcreb variants. Crosses between α-adaptin and the dCreb variants were performed, and an effect on viability of the hsCreb17-2 inhibitor was observed. The effect showed the same polarity, an increase in viability, as had been seen previously in its interaction with Appl^{d} (Table 3).

C. Behavioral Tests of Appl^{d} Interactions

Appl^{d} flies have a characteristic behavioral phenotype: a defect in fast phototaxis (Luo et al., supra, 1992). Appl^{d} flies are non-phototactic, and the phenotype is fully recessive. Thus, Appl^{d}/+ flies are phenotypically normal (see Table 7). Fast phototaxis was assayed as described in Benzer, Proc. Natl. Acad. Sci. USA 58:1112-1119 (1967), on flies aged 3-5 days. Flies were analyzed to determine whether the normal phototaxis of flies with one dose of Appl⁺ could be made abnormal in combination with the loss of one dose from an interacting loci. Conversely, flies were analyzed to determine whether the defective phototaxis in flies with no functional Appl⁺ could be ameliorated by loss of one dose from one of these loci. Of the various loci shown to affect viability (Table 3), two showed significant phototaxis interactions with Appl^{d}/+ flies: Notch and Delta. Three showed significant phototaxis interactions with Appl^{d} flies: α-adaptin, dCrebB and dCrebA.

These results utilize a second phenotypic assay to independently confirm the involvement of several loci indicated to be part of an Alzheimer's disease network based on their effects on viability when combined with Appl^{d}.

TABLE 7
Phototaxis Tests of Appl with Other Loci

| | | Phototaxis | |
|---|---|---|---|
| | N | Score | S.E.M. |
| Females | | | |
| w Appl^{d}/w Appl^{d} | 2 | 1.95 | 0.46 |
| w Appl^{d}/+ | 2 | 5.16* | 0.16 |
| w Appl^{d}/+; Dl⁷/+ | 2 | 2.26 | 1.07 |
| Dl⁷/+ | 2 | 4.32* | 0.48 |
| w Appl^{d}/w N²⁶⁴⁻³⁹ | 5 | 2.53 | 0.29 |
| w/w N²⁶⁴⁻³⁹ | 3 | 4.20* | 0.32 |
| Males | | | |
| w App^{d}/Y | 2 | 2.07 | 0.14 |
| w Appl^{c}/Y; α-adaptin⁰⁶⁶⁹⁴/+ | 8 | 3.78* | 0.57 |
| w Appl^{d}/Y; hs-Creb 17-2/+ | 10 | 4.96* | 0.18 |
| w Appl^{d}/Y; CrebA⁰⁸⁵⁷⁶/+ | 2 | 4.81* | 0.40 |

Phototaxis tested as described in Benzer, supra, 1967, with 20-40 files per assay.
*indicates significant difference (P < 0.05) from control (w Appl^{d}/+ for Females, w Appl^{d}/Y for Males) by Dunnett's test.

All journal articles and references provided herein, in parenthesis or otherwise, are incorporated herein by reference.

Although the invention has been described with reference to the examples provided above, it should be understood that various modifications can be made without departing from the spirit of the invention. Accordingly, the invention is limited only by the claims.

We claim:

1. A method of mapping a network of functional gene interactions relating to Alzheimer's disease, comprising the steps of:
(a) performing matings between (1) a first parent strain carrying a mutation in said Alzheimer's disease gene and (2) a series of parent strains each containing one of a series of genetic variations
to produce a series of test progeny, each of said test progeny carrying a mutation in said Alzheimer's disease gene and one of said series of genetic variations; and
(b) screening said series of test progeny for an altered phenotype relative to at least one sibling control, thereby localizing a gene that is a member of an Alzheimer's disease genetic network to one of said series of genetic variations.

2. The method of claim 1, further comprising identifying said gene that is a member of an Alzheimer's disease genetic network.

3. The method of claim 1, further comprising iteratively repeating steps (a) and (b), thereby identifying a network of functional gene interactions relating to Alzheimer's disease.

4. The method of claim 1, wherein the Alzheimer's disease gene is amyloid precursor protein-like (Appl).

5. The method of claim 1, wherein the Alzheimer's disease gene is presenilin (Psn).

6. The method of claim 1, wherein the Alzheimer's disease gene is selected from the group consisting of har38, dCrebA, dCrebB, α-adaptin, garnet, shi, N, Su(H)1, Dl, man and bib.

7. The method of claim 1, wherein the series of genetic variations comprises at least twenty individual genetic variations.

8. The method of claim 1, wherein the series of genetic variations comprises at least one hundred individual genetic variations.

9. The method of claim 1, wherein the series of genetic variations comprises a genetic variation that maps to the X-chromosome.

10. The method of claim 1, wherein each of the genetic variations in the series maps to the X-chromo some.

11. The method of claim 1, wherein the series of genetic variations comprises a genetic variation that maps to an autosome.

12. The method of claim 1, wherein each of the genetic variations in the series maps to an autosome.

13. The method of claim 1, wherein each of the series of test progeny is doubly heterozygous for the mutation in the Alzheimer's disease gene and one of the series of genetic variations.

14. The method of claim 1, wherein at least one parental strain comprises a balancer chromosome.

15. The method of claim 1, wherein the parent strains are Drosophilidae.

16. The method of claim 15, wherein the parent strains are Drosophila melanogaster.

17. The method of claim 1, wherein the mutation in the Alzheimer's disease gene is selected from the group consisting of an amorph, hypomorph, antimorph, hypermorph and neomorph.

18. The method of claim 1, wherein the mutation in the Alzheimer's disease gene is a deficiency.

19. The method of claim 1, wherein the Alzheimer's disease gene maps to the X-chromosome.

20. The method of claim 1, wherein the Alzheimer's disease gene maps to an autosome.

21. The method of claim 1, wherein the phenotype is selected from the group consisting of viability, morphology and behavior.

22. A method of identifying a therapeutic agent for treating Alzheimer's disease, comprising the steps of:
(a) performing matings between a first parent strain carrying a mutation in an Alzheimer's disease gene and a second parent strain containing a genetic variation, whereby test progeny are produced,
wherein, in the absence of an agent, the parent strains produce test progeny having an altered phenotype relative to at least one sibling control;
(b) administering an agent to at least one strain selected from the group consisting of said first parent strain, said second parent strain and said test progeny; and
(c) assaying the test progeny for the altered phenotype,
wherein a modification of the altered phenotype producing a phenotype with more similarity to a wild type phenotype than the altered phenotype has to the wild type phenotype indicates that the agent is a therapeutic agent.

23. The method of claim 22, wherein said modification is a complete or partial reversion of the altered phenotype.

24. The method of claim 22, wherein the Alzheimer's disease gene is Appl.

25. The method of claim 22, wherein the Alzheimer's disease gene is Psn.

26. The method of claim 22, wherein the Alzheimer's disease gene is selected from the group consisting of har38, dCrebA, dCrebB, α-adaptin, garnet, shi, N, Su(H)1, Dl, man and bib.

27. The method of claim 22, wherein the parent strains are Drosophila melanogaster.

28. The method of claim 22, wherein the altered phenotype is increased viability.

29. The method of claim 22, wherein said altered phenotype is decreased viability.

30. An isolated nucleic acid molecule that is differentially expressed in Appl^{d} versus Appl⁺ Drosophila melanogaster, comprising a nucleic acid sequence having substantially the sequence of a nucleic acid sequence selected from the group consisting of SEQ ID NOS: 1 to 63.

31. The isolated nucleic acid molecule of claim 30, comprising a nucleic acid sequence selected from the group consisting of SEQ ID NOS: 1 to 63.

31. An isolated nucleotide sequence, comprising at least 10 contiguous nucleotides of a nucleic acid sequence selected from the group consisting of SEQ ID NOS: 1 to 63.

32. The isolated nucleotide sequence of claim 31, comprising at least 15 contiguous nucleotides of a nucleic acid sequence selected from the group consisting of SEQ ID NOS: 1 to 63.

33. An isolated nucleic acid molecule that is differentially expressed in Appl^{d} versus Appl⁺ Drosophila melanogaster, comprising a nucleic acid sequence having substantially the sequence of a nucleic acid sequence selected from the group consisting of SEQ ID NOS: 64 to 80.

34. The isolated nucleic acid molecule of claim 33, comprising a nucleic acid sequence selected from the group consisting of SEQ ID NOS: 64 to 80.

35. An isolated nucleotide sequence, comprising at least 10 contiguous nucleotides of a nucleic acid sequence selected from the group consisting of SEQ ID NOS: 64 to 80.

36. The isolated nucleotide sequence of claim 35, comprising at least 15 contiguous nucleotides of a nucleic acid sequence selected from the group consisting of SEQ ID NOS: 64 to 80.
